(19) United States
(12) Patent Application Publication
(10) Pub. No.: US 2020/0009071 A1
(43) Pub. Date: Jan. 9, 2020

(54) NEUTRALIZING VENOMOUS BIOMACROMOLECULES

(71) Applicant: THE REGENTS OF THE UNIVERSITY OF CALIFORNIA, Oakland, CA (US)

(72) Inventors: Kenneth J. Shea, Irvine, CA (US); Jeff O'Brien, Irvine, CA (US)

(21) Appl. No.: 16/469,769

(22) PCT Filed: Dec. 15, 2017
(86) PCT No.: PCT/US2017/066613

§ 371 Date: Jun. 14, 2019

Related U.S. Application Data
(60) Provisional application No. 62/435,559, filed on Dec. 16, 2016.
(60) Provisional application No. 62/472,277, filed on Mar. 16, 2017.
(60) Provisional application No. 62/472,266, filed on Mar. 16, 2017.

(51) Int. Cl. A61K 9/70; A61K 45/06; A61K 31/78; A61L 15/44; A61L 15/20; A61K 9/51; A61P 39/02

(52) U.S. Cl. CPC A61K 9/7023; A61K 45/06; A61K 31/78; A61L 15/44; A61L 2400/12; A61K 9/51; A61P 39/02; A61L 2300/434; A61L 15/20

(57) ABSTRACT

The present invention relates generally to compositions and methods comprising abiotic, synthetic polymer nanoparticles (NPs) with affinity and specificity to peptide toxins, enzymes, signaling proteins and other large biomacromolecules. The synthetic polymer NPs are an improvement over the current art due to insusceptibility to phospholipase attack, a mechanism common to many venoms. In one embodiment, the compositions and methods relate to synthetic polymer NPs with affinity and specificity to three finger toxins (3FTX) and phospholipase A2. In one embodiment, the compositions and methods are useful for delaying or preventing tissue necrosis due to envenomation.

### CROSS-REFERENCE TO RELATED APPLICATIONS

This application claims priority to U.S. Provisional Patent Application No. 62/435,559, filed Dec. 16, 2016, to U.S. Provisional Patent Application No. 62/472,266, filed Mar. 16, 2017, and to U.S. Provisional Patent Application No. 62/472,277, filed Mar. 16, 2017, the contents of which are each incorporated by reference herein in their entirety.

### STATEMENT REGARDING FEDERALLY SPONSORED RESEARCH OR DEVELOPMENT

This invention was made with government support under grant number DMR-1308363 awarded by the National Science Foundation, grant number T32GM108561 awarded by the National Institute of General Medical Sciences, and contract number W911NF-15-C-0068 awarded by the Defense Advanced Research Projects Agency. The government has certain rights in the invention.

### BACKGROUND OF THE INVENTION

Morbidity resulting from snake envenomation affects roughly 2.7 million people each year. Often, rapid local tissue necrosis from fast-acting toxins leads to amputations and other permanent handicaps. While losing a limb is debilitating regardless of the victim, it is especially consequential for the many agricultural workers in impoverished countries who are disproportionally affected by envenomation. If the victim is fortunate enough to be near a medical facility capable of treating the victim with serotherapy, the survival rate is considerably increased. However, immunoglobulin-based antivenom is often not sufficient to prevent fast-acting toxin proteins from doing substantial damage to tissue. In many cases, particularly in African countries such as Benin, Guinea, and Kenya, roughly 70-80% of snakebite victims are only treated by “traditional” practitioners. Their methods, which include cauterization, cryo-therapy, and the use of tourniquets, are often more harmful than they are therapeutic. Moreover, seeking traditional treatments requires time needed reach a medical hospital, which is often over 24 hours away in Africa, which prolongs the delay until treatment.

Administering antivenom in the field is considered a dangerous practice and is not recommended. Typically, antivenom is administered intravenously via a slow infusion in order to reduce potential immunogenic responses or early adverse reactions (EARs). This is unfortunately expected considering the immunoglobulins are non-humanized and are often of equine origin. If administered in the field, the antivenom must be injected intramuscularly, which substantially reduces the bioavailability of the antivenom (˜40%). Administration of antivenom therefore should only be considered if absolutely necessary.

In general, snakes from the Elapidae and Viperidae family are responsible for most snake bites and mortalities. Most venomous snakes from the Viperidae family produce substantial tissue necrosis. However, in the Elapidae family, snakes from the Naja genus produce significantly more morbid effects than other genera (Bungarus). Depending on the species, the tissue damage can have a number of different effects including: swelling, blistering, hemorrhage, and skeletal muscle death.

The protein families that are typically responsible for local tissue death are phospholipase A2 (mostly group II) and metalloproteinases (Zn²⁺). The metalloproteinases play a significant role in the spreading of venom toxins through proteolytic degradation of the extracellular matrix and therefore work synergistically with phospholipase A2 to induce necrosis (non-programmed cell death). However, the dermonecrosis caused by Naja venom is largely attributed to low molecular weight cytotoxins, a sub-class of the three-finger toxin (3FTX) family. This is a significant problem for treatment, because 3FTX are non-enzymatic proteins and therefore present more of a challenge to inhibit.

Currently, the only option available to treat and prevent morbidity is the use of serotherapy. This is problematic because it severely limits treatment options prior to arriving at a suitable medical facility. This is especially an issue for individuals in rural areas that are often disproportionately affected and do not have easy access to medical facilities.

Thus there is a need in the art for enhanced therapeutics to reduce or prevent the rate of tissue necrosis during envenomation. The present invention satisfies this unmet need.

### SUMMARY OF THE INVENTION

The present invention includes a bandage comprising a substrate layer; and a therapeutic layer comprising a nano-dote composition having at least one component selected from the group consisting of: N-isopropylacrylamide (NIPAm); N-phenylacrylamide (PAA); N-tert-butylacrylamide (TBAm); N,N′-methylenebisacrylamide (Bis); N-acryloyl L-Phenylalinine (APhe); and acrylic acid.

In one embodiment, the bandage comprises N-isopropylacrylamide (NIPAm); N-phenylacrylamide (PAA); N,N′-methylenebisacrylamide (Bis); and acrylic acid. In one embodiment, the bandage comprises between 20% and 30% NIPAm. In one embodiment, the bandage comprises between 30% and 50% PAA. In one embodiment, the bandage comprises between 10% and 20% Bis. In one embodiment, the bandage comprises between 10% and 30% acrylic acid. In one embodiment, the bandage comprises 25% NIPAm, 40% PAA, 15% Bis, and 20% acrylic acid.

In one embodiment, the bandage comprises a nano-dote composition comprising a nanoparticle. In one embodiment, the nano-dote composition has affinity to a venomous biomacromolecule. In one embodiment, the biomacromolecule is a three finger toxin (3FTX). In one embodiment, the biomacromolecule is phospholipase A2.

In one embodiment, the bandage further comprises at least one therapeutic agent. In one embodiment, the at least one therapeutic agent is an enzymatic inhibitor. In one embodiment, the enzymatic inhibitor is a metalloproteinase inhibitor. In one embodiment, the enzymatic inhibitor is a hyaluronidase inhibitor.

The present invention also includes a method of inhibiting, diminishing, or neutralizing the activity of a venomous biomacromolecule in a subject in need thereof, comprising administering to a subject the bandage of the present invention.

In one embodiment, the biomacromolecule is a three finger toxin (3FTX). In one embodiment, the biomacromolecule is phospholipase A2. In one embodiment, the administration is by topical application.

The present invention also includes a dispenser comprising: a body casing; a reservoir comprising a nano-dote composition having at least one component selected from the group consisting of: N-isopropylacrylamide (NIPAm); N-phenylacrylamide (PAA); N-tert-butylacrylamide (TBAm); N,N′-methylenebisacrylamide (Bis); N-acryloyl L-Phenylalinine (APhe); and acrylic acid; a plunger; and a hollow needle fluidly connected to the reservoir.

The present invention also includes a nano-dote composition comprising: N-i sopropylacrylamide (NIPAm); N-phenylacrylamide (PAA); N-tert-butylacrylamide (TBAm); N,N′-methylenebisacrylamide (Bis); N-acryloyl L-Phenylalinine (APhe); and acrylic acid.

In one embodiment, the composition comprises N-isopropylacrylamide (NIPAm); N-phenylacrylamide (PAA); N,N′-methylenebisacrylamide (Bis); and acrylic acid. In one embodiment, the composition comprises between 20% and 30% NIPAm. In one embodiment, the composition comprises between 30% and 50% PAA. In one embodiment, the composition comprises between 10% and 20% Bis. In one embodiment, the composition comprises between 10% and 30% acrylic acid. In one embodiment, the composition comprises 25% NIPAm, 40% PAA, 15% Bis, and 20% acrylic acid.

In one embodiment, the composition is a nanoparticle. In one embodiment, the composition has affinity to a venomous biomacromolecule. In one embodiment, the biomacromolecule is a three finger toxin (3FTX). In one embodiment, the biomacromolecule is phospholipase A2.

In one embodiment, the composition further comprises at least one therapeutic agent. In one embodiment, the at least one therapeutic agent is an enzymatic inhibitor. In one embodiment, the enzymatic inhibitor is a metalloproteinase inhibitor. In one embodiment, the enzymatic inhibitor is a hyaluronidase inhibitor.

The present invention also includes a method of inhibiting, diminishing, or neutralizing the activity of a venomous biomacromolecule in a subject in need thereof, comprising administering to a subject a therapeutically effective amount of the nano-dote composition of the present invention.

In one embodiment, the biomacromolecule is a three finger toxin (3FTX). In one embodiment, the biomacromolecule is phospholipase A2. In one embodiment, the administration is by topical application.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts an exemplary therapeutic bandage of the present invention.

FIG. 2 depicts the 1H nuclear magnetic resonance (NMR) spectrum of synthesized N,N′-(1,4-phenylene)bisacylamide (PheBis).

FIG. 3 depicts the 1H NMR spectrum of synthesized N-acryloyl L-Phenylalanine (APhe).

FIG. 4A and FIG. 4B list the composition and yields for the first generation batch of nanoparticles (FIG. 4A) and the results of characterizing the first generation batch of nanoparticles (FIG. 4B).

FIG. 5A and FIG. 5B depict the results of experiments investigating erythrocyte lysis without (FIG. 5A) and with (FIG. 5B) the addition of 100 μg/mL phosphatidylcholine at various venom concentrations. Bungarus caeruleus (blue), Naja sputatrix (red), Crotalus atrox (green). The data is normalized against a triton-X (detergent) control.

FIG. 6A and FIG. 6B depict the results of experiments investigating erythrocyte lysis for NPs 1-12 at 0.1 μg/mL (FIG. 6A) and 0.5 μg/mL (FIG. 6B) incubated with Bungarus caeruleus venom (1 μg/mL) and phosphatidylcholine (100 μg/mL). Control (+, red) represents Venom incubated without NPs, Control (−, yellow) represents samples without venom or NPs, and Control (TX, black) represents red blood cells (RBCs) incubated with the detergent triton-X. The data is normalized against the triton-X (detergent) control.

FIG. 7A and FIG. 7B depict the results of experiments investigating erythrocyte lysis observed for NPs 1-12 at 0.5 μg/mL incubated with (FIG. 7A) and without (FIG. 7B) phosphatidylcholine (100 μg/mL). Control (-) represents samples without NPs, and Control (TX, black) represents RBCs incubated with the detergent triton-X. The data is normalized against the triton-X (detergent) control.

FIG. 8A depicts the results of an erythrocyte lysis assay for NP 1_5 (500 μg/mL) incubated with lysophosphatidylcholine (solid blue) versus lysophosphatidylcholine incubated without NPs (striped blue) at various concentrations of lysophosphatidylcholine.

FIG. 8B depicts the monomer feed ratio for NP 1_5.

FIG. 9A and FIG. 9B list the composition and yields for the second generation batch of nanoparticles (FIG. 9A) and the results of characterizing the second generation batch of nanoparticles (FIG. 9B).

FIG. 10 illustrates a PLA2 activity assay used to analyze inhibition of enzymatic hydrolysis of phosphatidylcholine to lysophosphatidylcholine (hemolytic).

FIG. 11 depicts the results of experiments investigating erythrocyte lysis without the addition of phosphatidylcholine, analyzing the onset of melittin hemolytic activity.

FIG. 12A and FIG. 12B depict the results of experiments investigating erythrocyte lysis without (FIG. 12A) and with (FIG. 12B) phosphatidylcholine using 10 μg/mL of whole Bee venom and a final concentration of 0.5 mg/mL NP 2_1, 2_2, 2_3, 2_4, and 2_12.

FIG. 13A and FIG. 13B depict the results of experiments investigating erythrocyte lysis without (FIG. 13A) and with (FIG. 13B) phosphatidylcholine using 10 μg/mL of whole Bee venom and a final concentration of 0.5 mg/mL NP 2_5, 2_6, 2_7, 2_8, and 2_12.

FIG. 14A and FIG. 14B depict the results of experiments investigating erythrocyte lysis without (FIG. 14A) and with (FIG. 14B) phosphatidylcholine using 10 μg/mL of whole Bee venom and a final concentration of 0.5 mg/mL NP 2_9, 2_7, 2_10, 2_11, and 2_12.

FIG. 15A and FIG. 15B depict the results of experiments investigating erythrocyte lysis without (FIG. 15A) and with (FIG. 15B) phosphatidylcholine using 10 μg/mL of whole Bee venom and varying concentrations of NP2_11.

FIG. 16A depicts the results of experiments investigating erythrocyte lysis without phosphatidylcholine using 10 μg/mL of whole Bee venom and varying concentrations of NP2_12.

FIG. 16B depicts the monomer feed ratio for NP 2_12.

FIG. 17 depicts the results of an MTT assay examining K562 survival rate in the presence of NP 2_12 at various concentrations. The results were normalized to results incubated without NP 2_12. The survival rate is equal to the mean (+/−) the standard deviation (n=6).

FIG. 18A illustrates the strategy for analyzing selectivity for targeted venom proteins over human serum proteins. Serum was diluted to a final concentration of 25%, PLA2 was diluted to a final concentration of 250 μg/mL, and NP 2_12 was diluted to a final concentration of 1 mg/mL.

FIG. 18B depicts the displacement of serum protein of a NP corona by venom proteins to effect in vivo venom sequestration/neutralization.

FIG. 18C is an unstained TEM image of NP 2_12. Scale bar=200 nm.

FIG. 19A through FIG. 19C depict the results of SDS-PAGE gel analysis investigating the selectivity for PLA2 from Naja mossambica venom (˜1% w/w total protein) in ovine plasma (25%) (FIG. 19A), Apis meliffera venom (˜1% w/w total protein) in ovine plasma (25%) (FIG. 19B), and Naja mossambica venom (˜1% w/w total protein) in human serum (25%) (FIG. 19C) using NP 2_12. The labelled bands in FIG. 17C were excised and subjected to proteomic analysis. (see FIG. 21A through FIG. 21C).

FIG. 20 depicts the results of whole venom selectivity experiments visualized by SDS-PAGE. (A/A′) molecular weight ladder. (B) Bungarus caeruleus whole venom. (C) Serum control. (D) NP 2_12 incubated in serum only. (E) NP 2_12 incubated in serum and Bungarus caeruleus whole venom. (F) Naja mossambica mossambica whole venom. (G) NP 2_12 incubated in serum and Naja mossambica mossambica whole venom.

FIG. 21A through FIG. 21C list the results of the digestion of the SDS-PAGE gel depicted in FIG. 19C.

FIG. 22 depicts the kinetic analysis of the dissociation of bee venom PLA2·NP 2_12 using NP 2_12 immobilized surface plasmon resonance (SPR) under flow conditions.

FIG. 23 illustrates the strategy for analyzing selectivity for targeted venom proteins over human serum proteins and the NP composition used in the venom selectivity experiments.

FIG. 24 depicts the SDS-PAGE results of an NP selectivity experiment for Bungaris fasciatus venom and Naja haje venom using 1% (w/w) venom in 25% human serum.

FIG. 25 depicts the SDS-PAGE results of an NP selectivity experiment for Naja nivea venom and Naja melonoleuca venom using 1% (w/w) venom in 25% human serum.

FIG. 26 depicts the SDS-PAGE results of an NP selectivity experiment for Naja sputatrix venom and a human serum control using 1% (w/w) venom in 25% human serum.

FIG. 27 depicts the SDS-PAGE results of an NP selectivity experiment for Dendroaspis polylepsis venom and Bitis arietans venom using 1% (w/w) venom in 25% human serum.

FIG. 28 depicts the SDS-PAGE results of an NP selectivity experiment for Naja mossambica venom and a human serum control using 1% (w/w) venom in 25% human serum.

FIG. 29 depicts the SDS-PAGE results of an NP selectivity experiment for Bungaris caeruleus venom and a human serum control using 1% (w/w) venom in 25% human serum.

FIG. 30 depicts the gel bands selected for trypsin digestion and LC-MS/MS proteomics analysis for the Dendroaspis polylepsis venom and Bitis arietans venom selectivity experiments from FIG. 26.

FIG. 31 depicts the gel bands selected for trypsin digestion and LC-MS/MS proteomics analysis for the Naja mossambica venom selectivity experiments from FIG. 27.

FIG. 32 depicts the gel bands selected for trypsin digestion and LC-MS/MS proteomics analysis for the Bungaris caeruleus venom selectivity experiments from FIG. 28.

FIG. 33 is a table listing the toxins identified in a Naja mossambica venom experiment.

FIG. 34 is a table listing the toxins identified in a Bungarus caeruleus venom experiment. #PTs=number of peptides.

### DETAILED DESCRIPTION

The present invention relates generally to compositions and methods comprising abiotic, synthetic polymer nanoparticles (NPs) with affinity and specificity to peptide toxins, enzymes, signaling proteins and other large biomacromolecules. The synthetic polymer NPs are an improvement over the current art due to insusceptibility to phospholipase attack, a mechanism common to many venoms. In one embodiment, the compositions and methods relate to synthetic polymer NPs with affinity and specificity to three finger toxins (3FTX) and phospholipase A2. In one embodiment, the compositions and methods are useful for delaying or preventing tissue necrosis due to envenomation.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “treating” means ameliorating the effects of, or reducing, delaying, halting, reversing, diminishing, or eliminating the frequency or the occurrence or the severity of at least one sign or symptom of an affliction, disease, or disorder.

As used herein, the terms “effective amount” or “therapeutically effective amount” or “pharmaceutically effective amount” of a composition are used interchangeably to refer to the amount of the composition that is sufficient to provide a beneficial effect to the subject to which the composition is administered. An appropriate therapeutic amount in any individual case may be determined by one of ordinary skill in the art using routine experimentation.

As used herein, a “therapeutic” treatment is a treatment administered to an individual who exhibits signs or symptoms of a disease or disorder for the purpose of ameliorating the effects of, or reducing, delaying, halting, reversing, diminishing, or eliminating the frequency or occurrence or the severity of those signs or symptoms.

As used herein, the term “pharmaceutically acceptable” refers to a material, such as a carrier or diluent, that does not abrogate the biological activity or properties of a compound and is relatively non-toxic; i.e., the material may be administered to an individual without causing undesirable biological effects or interacting in a deleterious manner with any of the components of a composition in which it is contained.

As used herein, a “pharmaceutically acceptable carrier” means a pharmaceutically acceptable material, composition, or carrier, such as a liquid or solid filler, diluent, excipient, solvent, or encapsulating material, involved in carrying or transporting at least one compound of the present invention within or to the subject such that it can perform its intended function. Each carrier must be “acceptable” in the sense of being compatible with the other ingredients of the formulation, and not be injurious to the patient. Some examples of materials that can serve as pharmaceutically acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, absorption delaying agents, and the like that are compatible with the activity of a compound and are physiologically acceptable to a subject. Supplementary active compounds can also be incorporated into the compositions.

As used herein, the term “nano-dote” or “nanodote” refers to a composition comprising one or more antivenom compounds of the present invention. The antivenom compounds include one or more synthetic polymer nanoparticles of the present invention having affinity and specificity to peptide toxins. The composition can include one or more therapeutic described herein. The composition can include one or more carrier described herein.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

The present invention provides nano-dote compositions comprising synthetic polymer nanoparticles (NPs) with affinity and specificity to peptide toxins, enzymes, signaling proteins and other large biomacromolecules. In certain embodiments, the invention comprises synthetic polymers with affinity and specificity to three finger toxins (3FTX) and phospholipase A2. In certain embodiments, the invention is effective in delaying or preventing tissue necrosis due to envenomation.

The present invention is partly based upon the discovery that synthetic polymer NPs comprising N-isopropylacrylamide (NIPAm), N, N′-methylenebisacrylamide (Bis), acrylic acid, N-tert-butylacrylamide (TBAm), N-phenylacrylamide (PAA), and N-acryloyl L-Phenylalanine (APhe) having binding affinity to certain venom proteins. Thus, the synthetic polymer NPs are able to bind to venom proteins and reduce or prevent destructive symptoms caused by their presence.

In certain embodiments, the synthetic polymer NPs may be described by the ratio of their components. For example, in certain embodiments, the synthetic polymer NPs may comprise between 20% and 90% NIPAm. In certain embodiments, the synthetic polymer NPs may comprise between 0% and 20% Bis. In certain embodiments, the synthetic polymer NPs may comprise between 0% and 50% acrylic acid. In certain embodiments, the synthetic polymer NPs may comprise between 0% and 50% TBAm. In certain embodiments, the synthetic polymer NPs may comprise between 0% and 50% PAA. In certain embodiments, the synthetic polymer NPs may comprise between 0% and 50% APhe. In one embodiment, the synthetic polymer NPs comprise 25% NIPAm, 15% Bis, 20% acrylic acid, and 40% PAA.

The synthetic polymer NPs described herein may be prepared in any suitable manner. Suitable synthetic methods used to produce the synthetic polymer NPs include, by way of non-limiting example, cationic, anionic, and free radical polymerization. In some instances, when a cationic process is used, a catalyst is used to initiate the polymerization. Optionally, one or more monomers may be used to form a copolymer. In some embodiments, the catalyst includes, e.g., protonic acids (Bronsted acid) or Lewis acids; in the case of using Lewis acids some promoter such as water or alcohols are also optionally used. In some embodiments, the catalyst is, by way of non-limiting example, hydrogen iodide, perchloric acid, sulfuric acid, phosphoric acid, hydrogen fluoride, chlorosulfonic acid, methansulfonic acid, trifluoromehtanesulfonic acid, aluminum trichloride, alkyl aluminum chlorides, boron trifluoride complexes, tin tetrachloride, antimony pentachloride, zinc chloride, titanium tetrachloride, phosphorous pentachloride, phosphorus oxychloride, or chromium oxychloride. In certain embodiments, polymer synthesis is performed neat or in any suitable solvent. Suitable solvents include, but are not limited to, pentane, hexane, dichloromethane, chloroform, or dimethyl formamide (DMF). In certain embodiments, the polymer synthesis is performed at any suitable reaction temperature, including, e.g., from about −50° C. to about 100° C., or from about 0° C. to about 70° C.

In certain embodiments, the synthetic polymers are prepared by free radical polymerization. When a free radical polymerization process is used, the monomer, optionally, the co-monomer, and an optional source of free radicals are provided to trigger the free radical polymerization process. In some embodiments, the source of free radicals are optional because some monomers may self-initiate upon heating at high temperature. In certain instances, after forming the polymerization mixture, the mixture is subjected to polymerization conditions. Polymerization conditions are those conditions that cause at least one monomer to form at least one polymer, as discussed herein. Such conditions are optionally varied to any suitable level and include, by way of non-limiting example, temperature, pressure, atmosphere, ratios of starting components used in the polymerization mixture, and reaction time. The polymerization is carried out in any suitable manner, including, e.g., in solution, dispersion, suspension, emulsion, or bulk.

Polymerization processes described herein optionally occur in any suitable solvent or mixture thereof. Suitable solvents include water, alcohol (e.g., methanol, ethanol, n-propanol, isopropanol, butanol), tetrahydrofuran (THF) dimethyl sulfoxide (DMSO), dimethylformamide (DMF), acetone, acetonitrile, hexamethylphosphoramide, acetic acid, formic acid, hexane, cyclohexane, benzene, toluene, dioxane, methylene chloride, ether (e.g., diethyl ether), chloroform, and ethyl acetate. In one aspect, the solvent includes water, and mixtures of water and water-miscible organic solvents such as DMF.

The synthetic polymer NPs can have any suitable size. NP sizes may be adjusted to meet specific needs by adjusting the proportion of components contained therein. In specific embodiments, the NP provided herein have an average hydrodynamic diameter of about 50 nm to about 150 nm. In other embodiments, the NP provided herein have an average hydrodynamic diameter of about 1 nm to about 500 nm, about 5 nm to about 250 nm, about 10 nm to about 200 nm, about 10 nm to about 100 nm, about 20 nm to about 100 nm, about 30 nm to about 90 nm, and the like. Particle size can be determined in any suitable manner, including, but not limited to, by gel permeation chromatography (GPC), dynamic light scattering (DLS), electron microscopy techniques (e.g., TEM), and other methods.

### Therapeutic Agents

Provided in certain embodiments herein is a NP comprising the abiotic, synthetic polymers of the present invention and at least one therapeutic agent. The NP is capable of binding to venom proteins, and is thereby administered in proximity to tissue damaged by the venom proteins and amenable to treatment by at least one therapeutic agent. The therapeutic agent can include any naturally occurring, synthetic, inorganic, organic, peptide, enzyme, nucleic acid small molecule, and the like, which has at least some activity in treating and/or preventing cancer.

In one embodiment, the at least one therapeutic agent comprises an enzymatic inhibitor. In certain embodiments, the therapeutic agent inhibits metalloproteinases and hyaluronidases, proteins that contribute to the spread of toxins. Non-limiting examples include tanomastat, prinomastat, batimastat, marimastat, doxycycline, bisphosphonates, heparin, gossypol, fenoprofen, disodium cromoglycate, tranilast, tetradecane sulfonic acid, glycerrhizic acid, sodium aurothiomalate, and the like.

In other embodiments, the present invention is not limited to any particular therapeutic agent, but rather encompasses any suitable therapeutic agent that can be embedded within a NP. Exemplary therapeutic agents include, but are not limited to, anti-viral agents, anti-bacterial agents, anti-inflammatory agents, antiseptics, anesthetics, analgesics, pharmaceutical agents, small molecules, peptides, nucleic acids, and the like.

In certain embodiments, the NP described herein comprise at least one antibacterial agent. In one embodiment, the antibacterial agent is a broad-spectrum antibacterial agent. Suitable antibacterial agents include, but are not limited to, chlorhexidine and derivatives thereof, members of the bisbiguanide class of inhibitors, povidone iodine, hydrogen peroxide, doxycycline, minocycline, clindamycin, doxycycline, metronidazole, essential oil extracts (menthol, thymol, eucalyptol, methyl salicylate, metal salts (zinc, copper, stannous ions), phenols (triclosan), all quaternary ammonium compounds (cetylpyridinium chloride), surfactants (sodium lauryl sulphate, delmopinol), all natural molecules (phenols, phenolic acids, quinones, alkaloids, lectins, peptides, polypeptides, indole derivatives, flustramine derivatives, carolacton, halogenated furanones, oroidin analogues, agelasine, ageloxime D).

In various embodiments, the at least one therapeutic agent is attached to the NP in any suitable manner. For example, attachment may be achieved through covalent bonds, non-covalent interactions, static interactions, hydrophobic interactions, or combinations thereof.

In certain embodiments, the nano-dote composition comprises at least one antibacterial agent. In one embodiment, the antibacterial agent is a broad-spectrum antibacterial agent. Suitable antibacterial agents include, but are not limited to, chlorhexidine and derivatives thereof, members of the bisbiguanide class of inhibitors, povidone iodine, hydrogen peroxide, doxycycline, minocycline, clindamycin, doxycycline, metronidazole, essential oil extracts (menthol, thymol, eucalyptol, methyl salicylate, metal salts (zinc, copper, stannous ions), phenols (triclosan), all quaternary ammonium compounds (cetylpyridinium chloride), surfactants (sodium lauryl sulphate, delmopinol), all natural molecules (phenols, phenolic acids, quinones, alkaloids, lectins, peptides, polypeptides, indole derivatives, flustramine derivatives, carolacton, halogenated furanones, oroidin analogues, agelasine, ageloxime D).

In various embodiments, the at least one therapeutic agent in the nano-dote composition is attached to the synthetic polymer NP. Attachment may be in any suitable manner. For example, attachment may be achieved through covalent bonds, non-covalent interactions, static interactions, hydrophobic interactions, or combinations thereof.

In some embodiments, therapeutic agents are selected from, by way of non-limiting example, at least one nucleotide (e.g., a polynucleotide), at least one carbohydrate or at least one amino acid (e.g., a peptide). In specific embodiments, the therapeutic agent is a polynucleotide, an oligonucleotide, a gene expression modulator, a knockdown agent, an siRNA, an RNAi agent, a dicer substrate, an miRNA, an shRNA, an antisense oligonucleotide, or an aptamer. In other embodiments, the therapeutic agent is an aiRNA (Asymmetric RNA duplexes mediate RNA interference in mammalian cells. Xiangao Sun, Harry A Rogoff, Chiang J Li Nature Biotechnology 26, 1379-1382 (2008)).

In certain embodiments, the therapeutic agent is a protein, peptide, dominant-negative protein, enzyme, antibody, or antibody fragment. In some embodiments, the therapeutic agent is a carbohydrate, or a small molecule. In some embodiments, the therapeutic agent is an abiotic, synthetic polymer. In some embodiments, a therapeutic agent is chemically conjugated to the

NP and/or to one or more polymer of the NP by any suitable chemical conjugation technique. Therapeutic agents are optionally conjugated to an end of the polymer, or to a pendant side chain of the polymer. In some embodiments, NP containing a therapeutic agent are formed by conjugation of the agent with a polymer and subsequently forming the NP in any suitable manner, e.g., by self-assembly of the resulting conjugates into a NP comprising the agent. The covalent bond between a polymer and a therapeutic agent of a NP described herein is, optionally, non-cleavable, or cleavable. In some embodiments, conjugation is also performed with pH-sensitive bonds and linkers, including, but not limited to, hydrazone and acetal linkages. In certain embodiments, the nano-dote composition comprises synthetic polymer NPs having a therapeutically effective amount of at least one therapeutic agent. For example, in one embodiment, the core of the NPs are loaded with a therapeutically effective amount of at least one therapeutic agent. The relative amount or concentration of the therapeutic agent may be dependent upon the size of the NPs, type of therapeutic agent, condition to be treated or prevented, and the like. In one embodiment, the therapeutic agent is present at greater than about 0 wt %, or greater than about 5 wt %, or greater than about 10 wt %, or greater than about 15 wt %, or greater than about 20 wt %, or greater than about 30 wt %, or greater than about 50 wt %, or greater than about 75 wt %. For example, it is demonstrated herein that the NPs may loaded with an amount or concentration of a therapeutic agent that is much greater than its minimum effective concentration. Thus, the nano-dote composition is able to retain therapeutically effective amounts of a therapeutic agent within the NPs.

In certain embodiments, the nano-dote composition comprises a plurality of different NPs, each carrying a different therapeutic agent, thereby providing combination therapy. For example, in one embodiment, the nano-dote composition comprises a first NP, comprising an enzymatic inhibitor, and a second NP, comprising an antibacterial agent. In another embodiment, the nano-dote composition comprises a first NP, comprising an enzymatic inhibitor, a second NP, comprising an antibacterial agent, and a third NP, comprising an anti-inflammatory agent. Each therapeutic agent has different yet complementary mechanisms of action, all aimed at treating the pathology. In one embodiment, the different NPs are mixed in different proportions to achieve maximum therapeutic effect. In one embodiment, each of the different NPs can be configured for different drug delivery characteristics, thereby allowing different therapeutic agents to be delivered at different times, as necessitated by the particular disorder or treatment.

### Pharmaceutical Compositions and Formulations

The invention also encompasses the use of pharmaceutical or nano-dote compositions of the invention to practice the methods of the invention. Such a nano-dote composition may consist of at least one compound, agent, NP, or NP conjugate of the invention in a form suitable for administration to a subject, or the nano-dote composition may comprise at least one compound, agent, NP, or NP conjugate of the invention, and one or more pharmaceutically acceptable carriers, one or more additional ingredients, or some combination of these.

In an embodiment, the nano-dote compositions useful for practicing the methods of the invention may be administered to deliver a dose of between 1 ng/kg/day and 100 mg/kg/day. In another embodiment, the nano-dote compositions useful for practicing the invention may be administered to deliver a dose of between 1 ng/kg/day and 500 mg/kg/day.

The relative amounts of the active ingredient, the pharmaceutically acceptable carrier, and any additional ingredients in a nano-dote composition of the invention will vary, depending upon the identity, size, and condition of the subject treated and further depending upon the route by which the nano-dote composition is to be administered. By way of example, the nano-dote composition may comprise between 0.1% and 100% (w/w) active ingredient.

Nano-dote compositions that are useful in the methods of the invention may be suitably developed for oral, rectal, vaginal, parenteral, topical, pulmonary, intranasal, buccal, ophthalmic, or another route of administration. A nano-dote composition useful within the methods of the invention may be directly administered to the skin or any other tissue of a mammal. The route(s) of administration will be readily apparent to the skilled artisan and will depend upon any number of factors including the type and severity of the envenomation being treated, the type and age of the subject being treated, and the like.

The formulations of the nano-dote compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the nano-dote composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of nano-dote compositions provided herein are principally directed to nano-dote compositions that are suitable for ethical administration to humans, it will be understood by the skilled artisan that such nano-dote compositions are generally suitable for administration to animals of all sorts. Modification of nano-dote compositions suitable for administration to humans in order to render the nano-dote compositions suitable for administration to various animals is well understood, and the ordinarily skilled veterinary pharmacologist may design and perform such modification with merely ordinary, if any, experimentation. Subjects to which administration of the nano-dote compositions of the invention is contemplated include, but are not limited to, humans and other primates, mammals including commercially relevant mammals such as cattle, pigs, horses, sheep, cats, and dogs.

In one embodiment, the nano-dote composition is formulated using one or more pharmaceutically acceptable excipients or carriers. In one embodiment, the nano-dote composition comprises a therapeutically effective amount of a compound, agent, NP, or NP conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the nano-dote composition. Prolonged absorption of the injectable nano-dote compositions may be brought about by including in the nano-dote composition an agent that delays absorption, for example, aluminum monostearate or gelatin.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials.

The nano-dote composition of the invention may comprise a preservative from about 0.005% to 2.0% by total weight of the nano-dote composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In certain embodiments, the nano-dote composition includes an anti-oxidant and a chelating agent that inhibits degradation. Antioxidants include BHT, BHA, alpha-tocopherol, and ascorbic acid in the ranges of about 0.01% to 0.3% and BHT in the range of 0.03% to 0.1% of the total weight of the nano-dote composition. The chelating agent may be present in an amount of from 0.01% to 0.5% of the total weight of the nano-dote composition. Chelating agents include edetate salts (e.g. disodium edetate) and citric acid in the weight range of about 0.01% to 0.20% and in the range of 0.02% to 0.10% of the total weight of the nano-dote composition. The chelating agent is useful for chelating metal ions in the nano-dote composition that may be detrimental to the shelf life of the formulation. Other suitable and equivalent antioxidants and chelating agents may be substituted therefore as would be known to those skilled in the art.

### Bandages

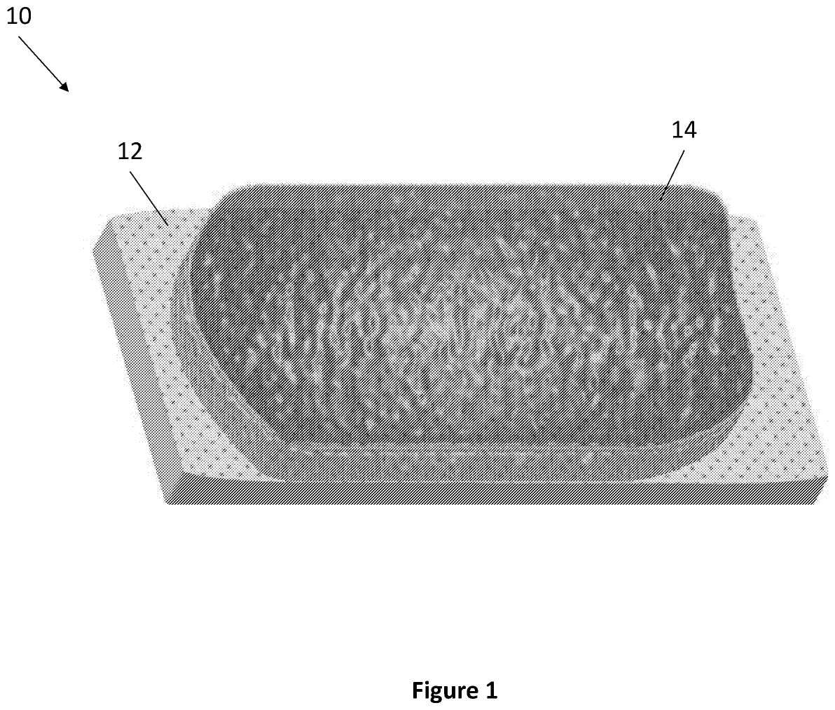

Referring now to FIG. 1, an exemplary bandage 10 is presented, comprising substrate layer 12 and therapeutic layer 14. Substrate layer 12 can comprise any suitable flexible substrate for holding therapeutic layer 14, such as a fabric or a plastic sheet. Substrate layer 12 can be sized so as to be applied to a single bite or sting, or it may be sized so as to be applied over a large envenomed area. Substrate layer 12 can be fabricated from thin and flexible materials, which enable bandage 10 to conform to the contours of the patient when applied thereon. Substrate layer 12 may be provided with an adhesive for enhanced attachment to a subject.

Therapeutic layer 14 can comprise any suitable substrate for holding nano-dote compositions, such as a hydrogel. Therapeutic layer 14 can have a smaller, a larger, or a similar size compared to substrate layer 12. Therapeutic layer 14 comprises one or more nano-dote compositions.

In certain embodiments, therapeutic layer 14 comprises one or more features to enhance delivery of nano-dote compositions. For example, therapeutic layer 14 can comprise a plurality of microneedles for transdermal penetration and delivery of nano-dote compositions. The microneedles can be coated with nano-dote compositions or have a hollow interior or cannula to dispense or inject nano-dote compositions. In some embodiments, the microneedles are embedded with nano-dote compositions and degrade or dissolve to deliver the nano-dote compositions.

It should be appreciated that the embodiments of the present invention are not limited to the depicted bandage 10. The therapeutic layer 14 comprising synthetic polymer NPs may be administered without a substrate layer 12, such as in the form of a cream, a lotion, a spray, a balm, and the like. Embodiments of the invention may also be suitably developed for oral, rectal, vaginal, parenteral, topical, pulmonary, intranasal, buccal, ophthalmic, and other routes of administration. Embodiments of the invention are also amenable to additional means of enhancing delivery, such as by jet injection via pressurized air or liquid, ballistic injection, ultrasound or acoustic treatment, iontophoresis, electroporation, thermal ablation, microdermabrasion, and the like.

### Dispensers

The dispensers of the present invention include delivery vehicles that are capable of providing immediate onsite intervention to mitigate the consequences of snake envenomation. In some embodiments, the dispensers are portable and lightweight such that the dispensers can be carried into the field without encumbering a user. In some embodiments, a dispenser comprises a plunger, a reservoir, a nano-dote composition stored within the reservoir, a hollow needle fluidly connected to the reservoir, and a body casing enclosing the components of the dispenser. The plunger is depressible to push the nano-dote composition through the hollow needle and out of the reservoir. In some embodiments, the hollow needle is stored in a retracted position within the body casing and is extended out of the body casing when the plunger is depressed.

In some embodiments, the plunger is depressed manually. In other embodiments, the plunger is depressed by a spring bias provided by at least one spring. The spring bias can be triggered manually, such as by a button, or the spring bias can be triggered mechanically, such as by a pressure-sensitive switch. For example, a pressure-sensitive spring bias can be triggered by impacting the dispenser against the epidermis of a user, whereupon the spring bias simultaneously extends the hollow needle to pierce the epidermis of the user and depresses the plunger to deliver the nano-dote composition to the user below the epidermis.

### Methods of Use

The present invention provides a method of treating or preventing tissue necrosis caused by envenomation. As described herein, the compositions described herein comprise synthetic polymer NPs having an affinity and specificity for certain venom proteins, such as 3FTX and phospholipase A2, wherein binding of the venom proteins to the synthetic polymer NPs lessen or prevent the amount of tissue destruction caused by envenomation. As such, the compositions comprising the synthetic polymers described herein are useful as antivenom agents.

The method of the invention can be used to treat any type of envenomation. Non-limiting examples include bites, stings, grazes, sprays, and the like. The treatment methods can be in any suitable form, including oral administration, parenteral administration, topical administration, and the like. Bandages and dispensers of the present invention can be applied directly to a site in need of treatment, such as a bee sting or snake bite, to deliver a steady amount of synthetic polymer NPs to sequester venom proteins and delay or prevent tissue necrosis.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: Engineering the Protein Corona of a Synthetic Polymer Nanoparticle for Broad-Spectrum Sequestration and Neutralization of Venomous Biomacromolecules

Snake envenomation is recognized by the World Health Organization (WHO) as a neglected tropical disease (Williams D et al., The lancet 375.9708 (2010): 89-91). Annually, 4.5 million people suffer from snakebites, 2.7 million suffer serious morbid injuries, and over 100,000 die as a result of snake envenomation (Gutiérrez J M et al., Toxicon 56.7 (2010): 1223-1235; Kasturiratne A R et al., PLoS Med 5 (2008): e218). The majority of the deaths occur in rural regions in South and Southeast Asia where individuals do not have immediate access to health care facilities capable of treating afflicted individuals (Alirol E et al., PLoS Negl Trop Dis 4.1 (2010): e603). In India alone, an estimated 35,000-50,000 people die annually from snake envenomation and 97% of these mortalities occur in rural regions (Mohapatra B et al., PLoS Negl Trop Dis 5.4 (2011): e1018).

Current methods of treating snake envenomation rely on the acquisition and administration of polyclonal antibodies from surrogate animals (Angulo Y et al., Toxicon 35.1 (1997): 81-90.). Unfortunately, in many areas, this approach has not been effective due to variations in venom composition that exist between different species and even populations within the same species (Fry B G et al., Toxicology and Applied Pharmacology 175.2 (2001): 140-148; Fry B G et al., Journal of Toxicology: Toxin Reviews 22.1 (2003): 23-34). The interspecific and intraspecific variability of snake venom has led many experts in the field to abandon the idea of creating a broad-spectrum antidote for snake venom (Fry B G et al., Journal of Toxicology: Toxin Reviews 22.1 (2003): 23-34). Moreover, the high cost of antivenom production has resulted in the discontinuation of African snake antivenom and coral snake antivenom from the world's leading suppliers (Hoffman R S, Clinical Toxicology 52.3 (2014): 157-159; Schiermeier Q, Nature 525.7569 (2015): 299-299). This is a major concern in Africa considering this region is home to many of the world's most venomous snake populations.

While there is a great deal of variability across snake venoms, there is also a rather striking level of homology (Reeks T A et al., Cellular and Molecular Life Sciences 72.10 (2015): 1939-1958). Venomous protein toxins can often be partitioned into a small number of protein families that are found abundantly in the venoms of numerous animals. For example, the protein family phospholipase A2 (PLA2) is found in many snake venoms, honey bee venoms, and scorpion venoms. Despite the fact that these families of protein toxins occur across multiple species, securing an effective antivenom is complicated by the diversity of protein isoforms found within each protein family. For example, over 300 different isoforms of PLA2 have been sequenced (Fry B G, Genome research 15.3 (2005): 403-420). PLA2, like many protein families found in venom, contains a large number of highly conserved disulfide bonds (Reeks T A et al., Cellular and Molecular Life Sciences 72.10 (2015): 1939-1958). This structural conservation and robust scaffold allows for a high degree of variation of exposed amino acid residues allosteric to the active site leading to diverse pharmacological profiles across the PLA2 protein family. This diversity on the primary sequence level is likely the reason why immunoglobulin based antivenoms are highly specific to particular venom compositions and often show poor cross-reactivity (Fry B G et al., Journal of Toxicology: Toxin Reviews 22.1 (2003): 23-34). Consequently, a challenge for any broad-spectrum antivenom is to develop an affinity reagent that recognizes features and similarities that are shared across venomous protein families but are unique with respect to abundant serum proteins to allow for selective sequestration.

The present study relates to the synthesis of abiotic protein/peptide affinity reagents, wherein non-toxic hydrogel copolymer nanoparticles (NPs) have been engineered with affinity and selectivity to peptide toxins, enzymes, signaling proteins and other large biomacromolecules (fibrinogen, IgG) through a directed synthetic evolution process (O'Brien J et al., Accounts of chemical research (2016)). These synthetic polymer NPs are analogous to biological protein affinity reagents (antibodies, aptamers, etc.). Examples include selective capture and programed release of lysozyme from complex protein mixtures (egg white) (Yoshimatsu K et al., Angewandte Chemie International Edition 51.10 (2012): 2405-2408), a NP with specificity for the Fc domain of IgG (Lee S et al., Journal of the American Chemical Society 134.38 (2012): 15765-15772), in vitro inhibition of PSMα3, an amphiphilic defensive peptide secreted by MRSA that disrupts cellular membranes, in the presence of serum proteins (Weisman A et al., Biomacromolecules 15.9 (2014): 3290-3295), and sequestration/neutralization of the peptide toxin melittin from the blood stream of a living mouse (Hoshino Y et al., Proceedings of the National Academy of Sciences 109.1 (2012): 33-38). More recently a polymer NP was developed with engineered affinity for a vascular endothelial growth factor (VEGF₁₆₅). The NP inhibits binding of the signaling protein to its receptor VEGFR-2, preventing receptor phosphorylation and downstream VEGF₁₆₅-dependent endothelial cell migration (Koide H et al., Nat Chem 2016 8).

NP selection process differs from that of the immune system. NP-protein affinity arises broadly from similarities that are characteristic of specific classes of toxins. PLA2 interacts with lipid membranes and lipoprotein particles (Gazi I et al., J. Hypertens. 2006, 24, S395). These features may be shared not only across isoforms but even different families of proteins. Hence, during the process of formulating and optimizing a NP capable of neutralizing venomous PLA2, the NP will also be able to sequester similar venomous proteins and peptides. Indeed, an ingenious use of biological membranes has been employed as a “toxin sponge” for toxins with membrane affinity (Hu, C J et al., Nature nanotechnology 8.5 (2013): 336-340). However, since PLA2, a membrane hydrolyzing enzyme, is a significant component of most snake venoms, this approach would not be successful. The synthetic polymer NPs of the present invention, which lack phospholipids, would not be vulnerable to PLA2-catalyzed degradation.

The materials and methods are now described.

### Materials

The following materials were obtained from commercial sources: N-isopropylacrylamide (NIPAm), ammonium persulfate (APS), 1,4-phenylenediamine, N-phenylacrylamide, acryloyl chloride, L-phenylalanine, Naja sputatrix venom, Bungarus caeruleus venom, and Crotalus atrox venom, Apis mellifera (Honey-bee) Phosphatidylcholine, Phospholipase A₂ from Honey-bee venom, Phospholipase A₂ from Naja mossambica venom, ovine plasma, human serum, and lysophosphatidylcholine were obtained from SIGMA-ALDRICH Inc.; sodium dodecyl sulfate (SDS) was obtained from Aldrich Chemical Company, Inc.; N,N′-methylenebisacrylamide (MBis) was from Fluka; N-tert-butylacrylamide (TBAm) was from ACRŌS ORGANICS. All other solvents and chemicals were obtained from Fisher Scientific Inc. or VWR International LLC. NIPAm was recrystallized from hexanes, and 1,4-phenylenediamine was sublimed before use.

Water used in polymerization and characterization was purified using a Barnstead Nanopure Diamond™ system. 12-14 kDa MWCO cellulose membranes were purchased from Spectrum Laboratories. Precast SDS-PAGE gels (4-15% Mini-Protean), Coomassie Brilliant Blue R-250 and molecular weight ladder (Precision plus protein standards) were purchased from Bio-rad Laboratories. Bovine Red Blood cells, Ovine plasma and human serum were purchased from Lampire Biological Laboratories.

### Instrumentation

UV-Vis absorption spectra were measured using a Thermo Scientific 2000c Nanodrop or a SpectraMax Plus 384 Microplate Reader. Nanoparticle size and polydispersity was determined using a Malvern ZEN3600 dynamic light scattering (DLS) instrument with a disposable sizing cuvette. Lyophilization of polymer samples was performed using a Labconco Freezone 4.5. ¹H NMR spectra were acquired on a Bruker DRX500 spectrometer with a TCI (three channel inverse) cryoprobe. All measurements were run at 298 K and were analyzed using the MestReNova (version: 6.0.2-5475) program. TEM Images were obtained on a FEI Tecnai G2 TF20 high resolution TEM operated at an accelerating voltage of 200 kV.

### Nanoparticle Synthesis

Nanoparticles were synthesized following a previously reported procedure (Weisman A et al., Biomacromolecules 15.9 (2014): 3290-3295). Monomers corresponding to the designated feed ratio, and SDS (30 mg, 1 μmol) were dissolved in water (50 mL) to a final monomer concentration of 65 mM. 2 mL of acetone was used to dissolve the aromatic monomers before adding to the bulk aqueous solution. The resulting solution was degassed with nitrogen for 30 min while stirring. Ammonium persulfate (30 mg dissolved in 1 mL H₂O) was added to the degassed solution, and the reaction mixture was heated to 60° C. under nitrogen for 3 h. The polymerization was quenched by exposing the reaction mixture to air, and the reaction mixture was transferred to a 12,000-14,000 MWCO membrane and dialyzed against an excess of deionized water (changed twice a day) for 4 d.

N,N′-(1,4-phenylene)bisacylamide (PheBis)

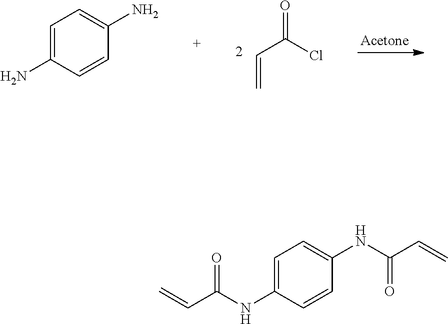

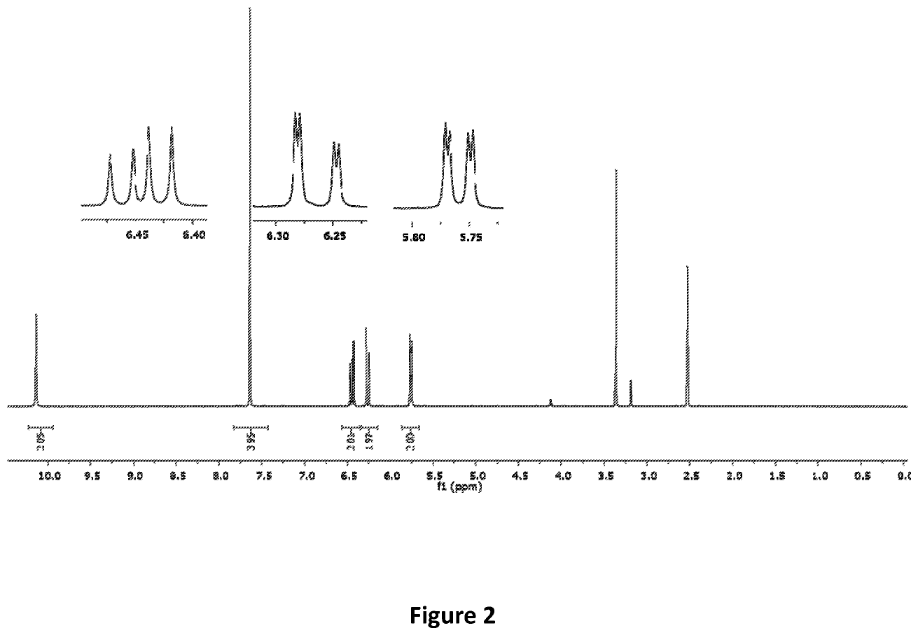

N,N′-(1,4-phenylene)bisacylamide was synthesized by the previously reported procedure with minor revisions (Al-Fulaij OA et al., Journal of applied polymer science 101.4 (2006): 2412-2422). 1,4-phenylenediamine (5.6 mmol, 605.6 mg) was mixed with acetone (25 mL), and cooled to 0° C. in an ice bath. Acryloyl chloride (11 mmol, 898.0 μL) was mixed with acetone (15 mL), and added dropwise to the solution of 1,4-phenylenediamine, while stirring, and allowed to warm to room temperature and kept at room temperature overnight. The solution was then washed with saturated NaHCO₃, filtered and the solid was washed with 100 mL of cold H₂O. The crude product was recrystallized from EtOH/MeOH, to afford white crystals that were collected by filtration (1.1890 g, 87%): ¹H NMR (500 MHz, DMSO): δ 10.14 (s, 2 H, (NH)₂), 7.64 (s, 4 H, (Phe-H)₄), 6.42 (dd, J=17.0, 10.0, 2 H, (═CH)₂), 6.26 (dd, J=17.0, 2.0 Hz, 2 H, (═CH₂)₂), 5.76 (dd, J=10.0, 2.5 Hz, 2 H, (═CH₂)₂; ESMS m/z calcd for C₁₂H₁₂N₂O₂Na (M+Na)⁺ 239.08, found 239.04; mp>275° C. (Lit. 243° C.) (FIG. 2).

### N-acryloyl L-Phenylalanine (APhe)

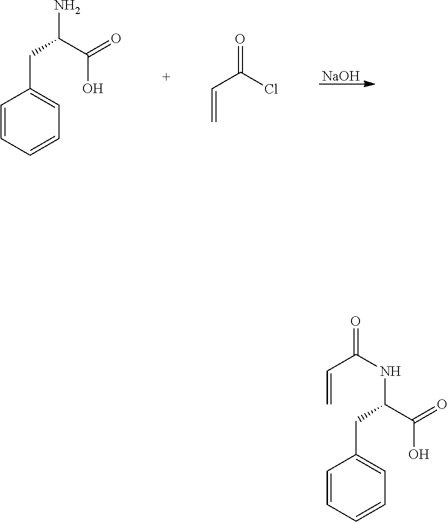

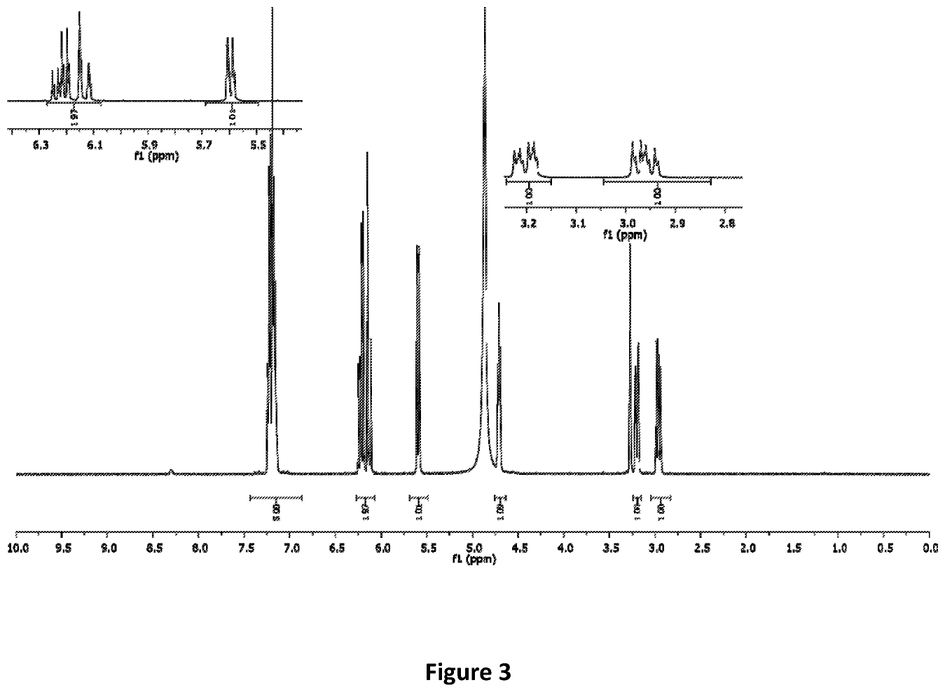

L-Phenylalanine (3.63 g, 0.020 mol) was dissolved in 20 mL of 2 M sodium hydroxide aqueous solution. After the solution became transparent, acryloyl chloride (1.75 mL, 0.022) was added dropwise with vigorous stirring while the reaction mixture was kept below 0° C. by external ice-bath cooling. After the addition was completed, the stirring was continued for an additional 2 h at room temperature and a white powder precipitated. The mixture was acidified to pH 2 with concentrated hydrochloride acid. The obtained white product was recrystallized with water (2.74 g, 45.7% yield):: ¹H NMR (500 MHZ, CD₃OD): δ7.24˜7.16 (m, 5 H, C₆H₅), 6.27˜6.13 (dd, J=17.5 Hz, 10.5 Hz, 1 H, CH═CH₂), 6.21˜6.13 (d, J=17.5 Hz, 1 H, CH═CH_{2 trans}), 5.62 (d, J=10.5 Hz, 1 H, CH═CH_{2 cis}), 4.75˜4.71 (m, CHCH₂), 3.25˜3.20 (m, 1 H, CH₂CH), 3.01˜2.96 (m, 1 H, CH₂CH); ESMS m/z calcd for C₁₂H₁₃NNaO₃ [M+Na]⁺ 242.2263, found 242.3; mp 122.5˜124° C. (FIG. 3).

### Characterization of Nanoparticles

Dynamic light scattering (DLS) was used to characterize the size and dispersity of the NPs. All NPs were measured in nanopure water at 25° C. Selected NPs were measured at various temperatures in water and 50 mM Tris buffer (150 mM NaCl, 10 mM CaCl₂, pH=7.50), at 37° C. All samples were allowed to equilibrate at the designated temperature for 200 seconds prior to each measurement and were measured at a scattering angle of 173°. Concentrations and yields were determined by lyophilizing a known volume of the purified NP solution and weighing the obtained dry polymer samples.

### Generation 1 NPs

Bungarus caeruleus venom hemolytic assay

Bovine red blood cells (RBCs; Lampire Biological Laboratories, Pipersville, Pa.) were washed with 50 mM Tris buffer (with 150 mM NaCl, 10 mM CaCl₂, pH=7.50), collected by centrifugation (10 min, 3000 rpm), and resuspended in Tris buffer. Washing was repeated until the supernatant was sufficiently clear after which A 10% RBC suspension was prepared in tris buffer. In a 96-well plate Bungarus caeruleus venom (1 μg/mL final concentration) was preincubated with NPs (0, 0.1, and 0.5 mg/mL final concentration) for 15 minutes at 37° C. After 15 min, phosphatidylcholine (100 μg/mL final concentration) followed by RBCs (5% final concentration) were mixed with the preincubated NP-venom solution and incubated at 37° C. for an additional 15 min. Samples were then centrifuged at 4000 rpm for 10 min, and the release of hemoglobin was quantified by measuring the absorbance of the supernatant at 415 nm (Aₛₐₘₚₗₑ). RBCs incubated with the detergent triton-X (1%) were used to normalize the absorbance values. All Samples were run in triplicate.

(Aₛₐₘₚₗₑ−A_{tris bufer})/(A_{triton-X}−A_{tris buffer})×100=% Erythrocyte lysis
Lysophosphatidylcholine assay

Bovine red blood cells were washed with tris buffer (with 150 mM NaCl, 10 mM CaCl₂, pH=7.50) and collected as previously described. In a microcentrifuge tube, lysophosphatidylcholine (0-100 μg/mL) was incubated with and without NP 1_5 (0.5 mg/mL) for 15 min at 37° C. After 15 min, RBCs (5% final concentration) were mixed with the preincubated NP-lysophosphatidylcholine solutions or the lysophosphatidylecholine-only solutions and incubated at 37° C. for an additional 15 min. Samples were then centrifuged at 4000 rpm for 10 min, and the release of hemoglobin was quantified by measuring the absorbance of the supernatant at 415 nm (Aₛₐₘₚₗₑ). RBCs incubated with the detergent triton-X (1%) were used to normalize the absorbance values. All Samples were run in triplicate.

(Aₛₐₘₚₗₑ−A_{tris bufer})/(A_{triton-X}−A_{tris buffer})×100=% Erythrocyte lysis

### Generation 2 NPs

Whole honey-bee venom hemolytic assay

PLA2 and melittin inhibition: the hemolytic assay protocol was modified for the honey bee-venom to allow for more rigorous testing. Briefly, honey-bee venom (10 μg/mL) was incubated with the second generation NPs (0.5 mg/mL, or as indicated) for 15 min (37° C.) in tris buffer (with 150 mM NaCl, 10 mM CaCl₂, pH=7.50). Next, phosphatidylcholine (100 μg/mL final concentration) followed by RBCs (5% final concentration) were mixed and incubated at 37° C. for 1 h. Results were normalized to the detergent control. All samples were run in triplicate and quantified as previously described.

Melittin inhibition: honey-bee venom (10 μg/mL) was incubated with the second generation NPs (0.5 mg/mL) for 15 min (37° C.) in tris buffer (with 150 mM NaCl, 10 mM CaCl₂, pH=7.50). Next, RBCs (5% final concentration) were mixed and incubated at 37° C. for 1 h. Results were normalized to the detergent control. All samples were run in triplicate and quantified as previously described.

### PLA₂ Selectivity in Human Serum

In a microcentrifuge tube, NP 2_12 (1 mg/mL final concentration) was incubated with human serum (25% final concentration) in phosphate buffer saline (Dulbecco's) for 5 min at 37° C. Next, phospholipase A₂ (250 μg/mL final concentration) was added to the NP 2_12/human serum mixture and incubated for an additional 45 min at 37° C. The solution was then centrifuged at 10,000 RPM for 10 min, and the supernatant was replaced with fresh phosphate buffer saline four consecutive times or until the supernatant was depleted of protein by SDS-PAGE (commassie blue stain). Next, 20 μL of SDS-PAGE sample preparation mixture was added to the NP pellet, mixed and heated to 95° C. for 10 minutes. Finally, the Mixture was centrifuged at 5,000 RPM and the bound proteins were analyzed by SDS-PAGE (100 V).

### Tryptic Digest of Gel Entrapped Proteins

Excised gel bands where washed in a 100 μL of a 50:50 of 100mM NH₄HCO₃/CH₃CN for 10 min. The supernatant was removed and 30 μL of CH₃CN was added for 10 min. The wash step was repeated until the gels appeared colorless (3×). Finally, the gels were placed in a vacuum oven (45° C.) to dry (5-10 min). Next, 10-20 of 10 mM DTT (DTT solution prepared using 50 mM NH₄HCO₃ buffer) and was incubated at 60° C. for 1 h. After cooling to RT, equal volume of 50 mM iodoacetic acid (prepared in 50 mM NH₄HCO₃) and incubated for 45 min at 45° C. in the dark. Next, the supernatant was discarded and 10-20 μL of 50 mM NH₄HCO₃ buffer to each excised gel band and incubate at RT for 10 min. The supernatant was removed and 50 μL of 100 mM NH₄HCO₃ was added for 5-10 min. The supernatant was removed and 30 μL of CH₃CN for added for 10 min. The NH₄HCO₃ and CH₃CN washes were then repeated twice, and finally the bands were dried for 5-10 minutes using a vacuum oven (45° C.) for 5-10 min. Next, 50 μL of porcine trypsin (50 mM NH₄HCO₃, 20 ng/μL) was added to each dried gel band for 45 min at 4° C., and then incubated at 37° C. overnight. Following, 20 μL of deionized H₂O to each gel band for 5 min. The supernatant was removed and transferred to a new microcentrifuge tube. 30 μL of 50% CH₃CN/1% TFA was added to each microcentrifuge tube containing the gel band for 10 min. After which, the supernatant was removed and transferred to the previous supernatant-only microcentrifuge tubes. The 50% CH₃CN/1% TFA wash was repeated 3 times. Finally, the supernatant tube was dried using a vacuum oven, and re-suspended in 50% CH₃CN/1% TFA.

### LCMS-MS Protocol

Following in-gel digestion with porcine trypsin, extracted peptides were separated on a BEH C18 nanotile column and analyzed by MSE on a SYNAPT G2 instrument with a triziac source (Waters). Data were analyzed using ProteinLynx Global Server software (PLGS 3.0) with the human proteome database in addition to the proteins found on www.uniprot.org from Naja mossambica venom. The LC gradient was linear 2% B to 95% B in 40 minute and the column was re-equilibrated for the next run. The MSE was performed by keeping the collision voltage at 6 eV to generate an MS spectra and later ramped up between 15-45 eV to generate the MS/MS spectra. The PLGS will process the MS and MS/MS data to accommodate the multiple peaks that were generated from multiple precursor ion and assign the appropriate peptide fragment for each ion.

### TEM Characterization

8 μL of NP 2_12 (1 mg/mL) was placed on a glow discharged TEM grid (Ultrathin Carbon Type-A, 400 mesh) and let stand for 1 min. The solution was removed by filter paper and the grid was left to air dry for 10 min. Next the grid was loaded into the instrument (FEI Tecnai G2 TF20 high resolution TEM operated at an accelerating voltage of 200 kV) for imaging.

### Surface Plasmon Resonance (SPR)

The NP immobilized SPRI chips were created by the modified procedure for SPRI from previous work (Weisman A et al., Biomacromolecules 15.9 (2014): 3290-3295). The Au coated SPRI chips were coated by thermally evaporated chromium (1 nm) as an adhesion layer and Au (45 nm) on SF10 glass slides (18×18 mm; Schott Glass). Before coating, those slides were cleaned by plasma cleaner (PDC-32G; Harrick Plasma) and then treated with Sigmacote (Manuel G et al., The Journal of Physical Chemistry C (2016)). The SPRI chips were rinsed with ethanol and nanopure water and dried by nitrogen before use. SPR measurements were carried out with SPR imager II (GWC Technologies, Madison, Wis.) at room temperature. The SPRI chip was flushed with PBS buffer at pH 7.4. After obtaining flat baseline, NP 2_12 was immobilized on the chip by using 0.5 mg/mL and 1.0 mg/mL of the NP samples in PBS buffer. The NP immobilization was monitored with SPRI. Next, bee-venom PLA₂ (20 μM) was introduced to the NP immobilized chip at RT. After saturation was reached, fresh PBS buffer was flowed over the PLA₂⋅NP immobilized SPRI chip. Data was normalized by fixing the maximum ΔR% at 100% capacity.

### Nanoparticle Toxicity Test-MTT Assay (Cell Viability Assay)

K562 cells were counted with a countess automated cell counter, centrifuged at 1500 rpm, redispersed in PBS (phosphate buffered Saline solution from Sigma) and 200,000 cells per well were seeded in a 96 well plate. A mixture of PBS 5× (30 μL), X mL of NP (final concentration: 100, 200, 300, 400 and 500 μg/mL), 120-X μL of nanopure water was prepared. Two controls were also prepared, one with no NPs, and one with only cells in PBS. 150 μL was added to the pelleted cells, mixed again with a multichannel pipet, and incubated for 30 min at 37° C. in a humidified 5% CO₂ incubator. The cells were pelleted by centrifugation at 3000 rpm and the supernatant was removed and replaced with 100 μL of fresh media (RPMI 1640 Medium including 10% fetal bovine serum and 1% Pen Strep from Gibco). The MTT (3-(4,5-Dimethylthiazol-2-yl)-2,5-Diphenyltetrazolium Bromide from molecular probe, 10 μL, 12 mM solution in PBS) regent was added and the wells mixed with a multichannel pipet. The mixtures were then incubated at 37° C. in a humidified 5% CO₂ incubator for 4 h, centrifuged at 3000 rpm, and all the media was replaced with DMSO (100 μL, Dimethylsulfoxide from ATCC). The solutions were incubated at 37° C. for 20 min, mixed and absorbance was read at 570 nm by Microplate reader (Bio-RAD).

The results are now described.

Venomous PLA2 enzymes are generally characterized by their low molecular weight (13-16 kDa), Ca²⁺-dependency, and a conserved active site histidine/aspartic acid dyad (Scott DL et al., Science 250.4987 (1990):1541). Unlike mammalian PLA2, which are relatively non-toxic and pharmacologically benign, venomous PLA2 are capable of producing a variety of pharmacological effects, making them one of the most toxic components of snake, honey-bee, and scorpion venom (Fry BG et al., Annual review of genomics and human genetics 10 (2009): 483-511; Kini RM, Toxicon 42.8 (2003): 827-840; Kini RM et al., Toxicon 27.6 (1989): 613-635). In the presence of phosphatidylcholine, PLA2 catalyzes the production of lysophosphatidylcholine, which is known to induce hemolysis (Bierbaum T J et al., Biochimica et Biophysica Acta (BBA)-Biomembranes 555.1 (1979): 102-110). This property allows for the activity of enzymatically active PLA2 isoforms to be monitored indirectly through hemolytic assays (Al-Abdulla I H et al., Toxicon 29.8 (1991): 1043-1046).

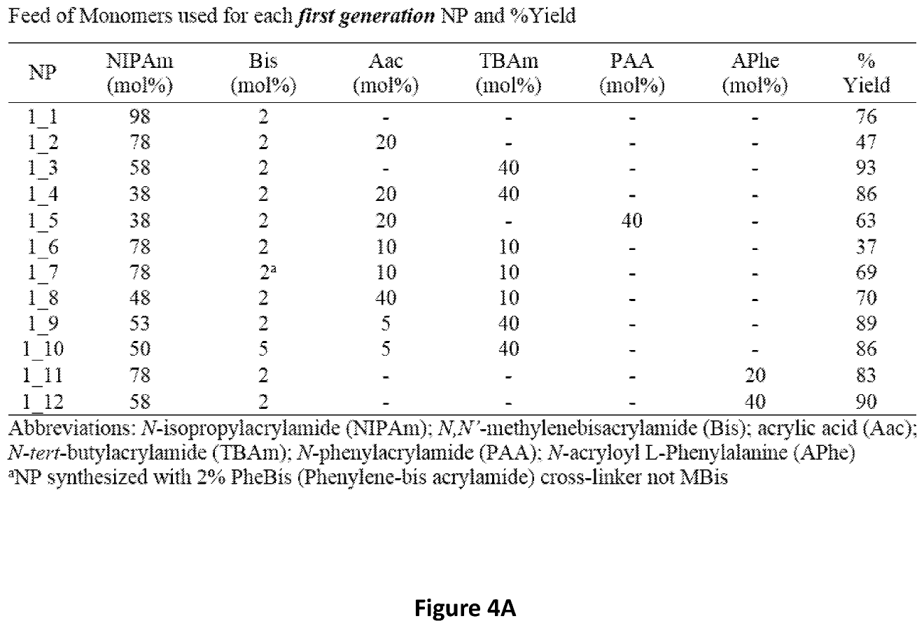

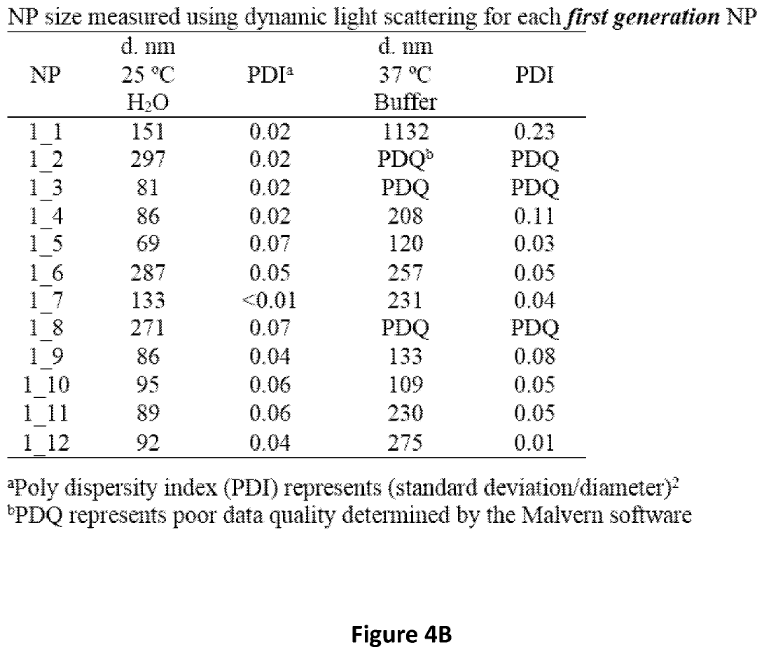

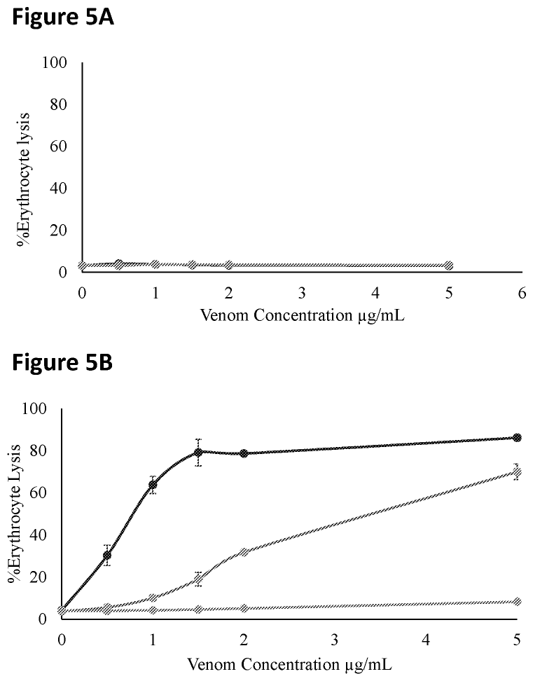

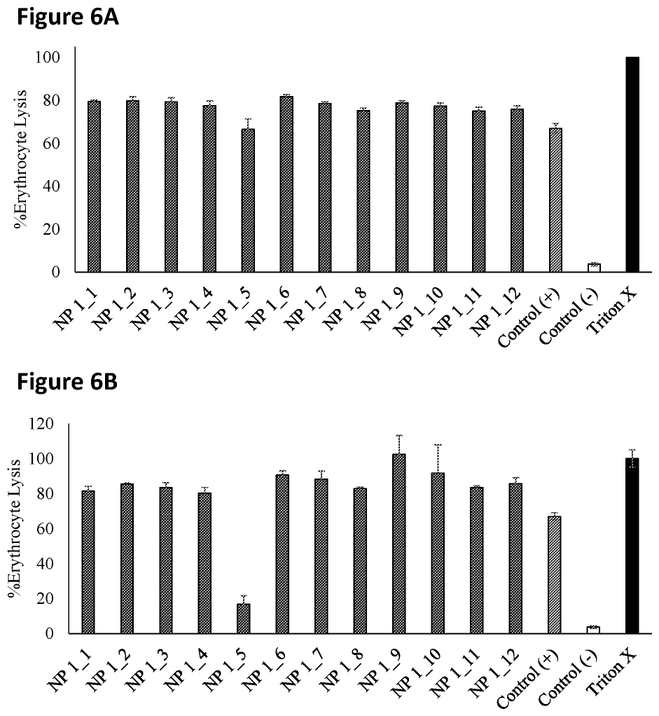

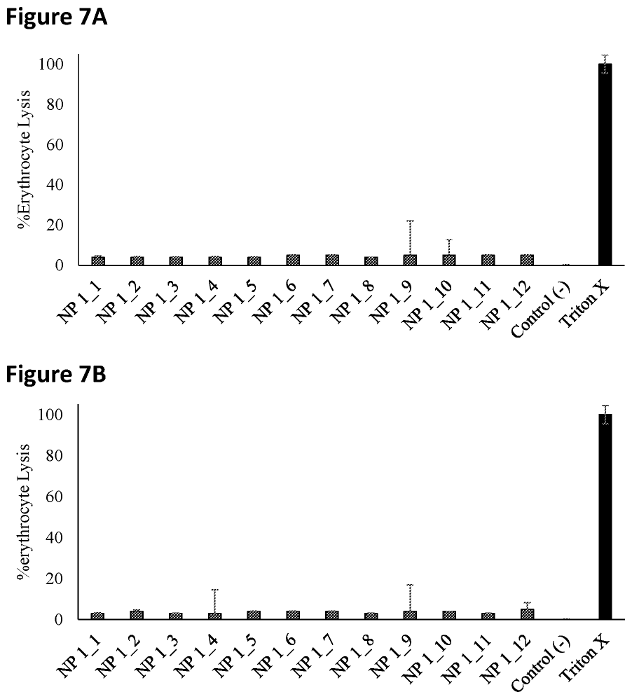

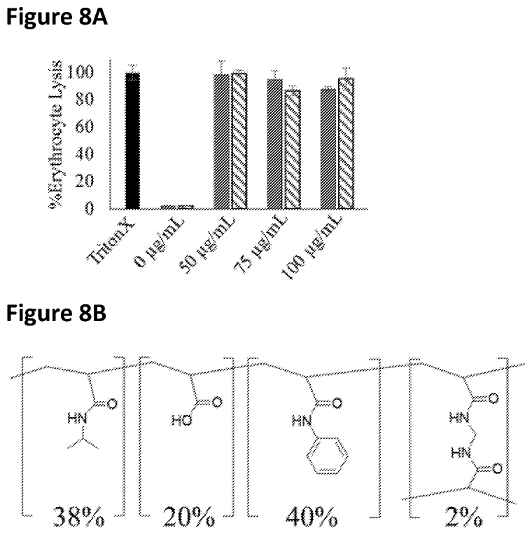

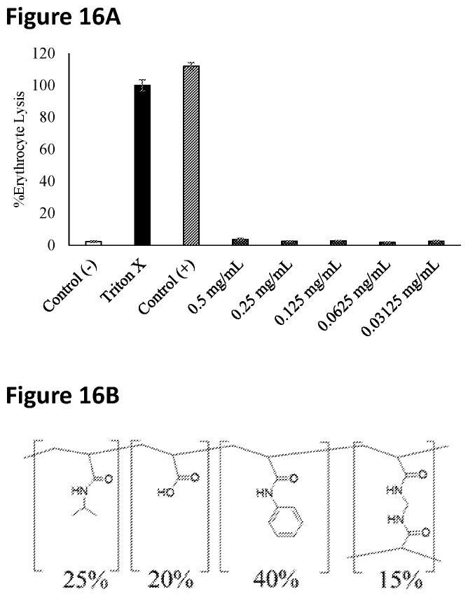

Using this assay, a first generation library (FIG. 4A, FIG. 4B) of functionally diverse NPs were analyzed for their ability to inhibit PLA2 induced erythrocyte lysis. It was discovered that NP 1_5, consisting of 20% acrylic acid, 40% N-phenylacrylamide, 38% N-isopropylacrylamide, and 2% N,N′-methylenebisacrylamide (FIG. 8B), showed a decrease in erythrocyte lysis when tested against Bungarus caeruleus (Indian Krait) venom (FIG. 6B). Furthermore, it appears that NP 1_5 does not prevent hemolysis by scavenging the lysophosphoatidylcholine (FIG. 16A). Rather, it is likely inhibiting the production of lysophosphatidylcholine by directly interacting with PLA2.

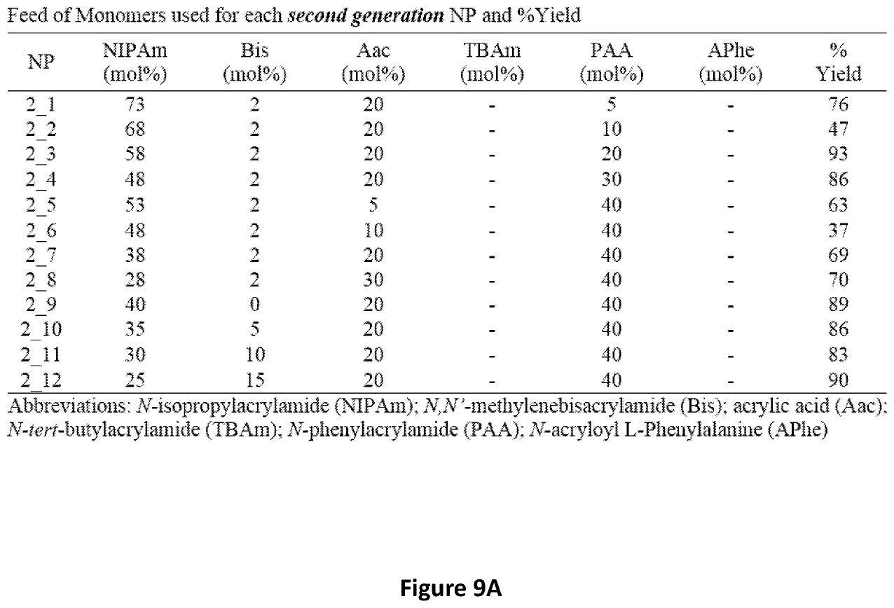

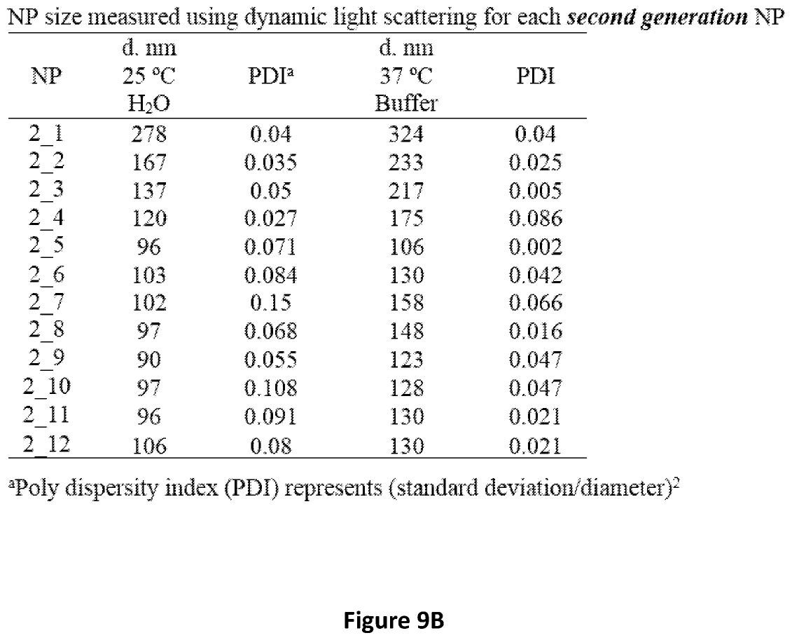

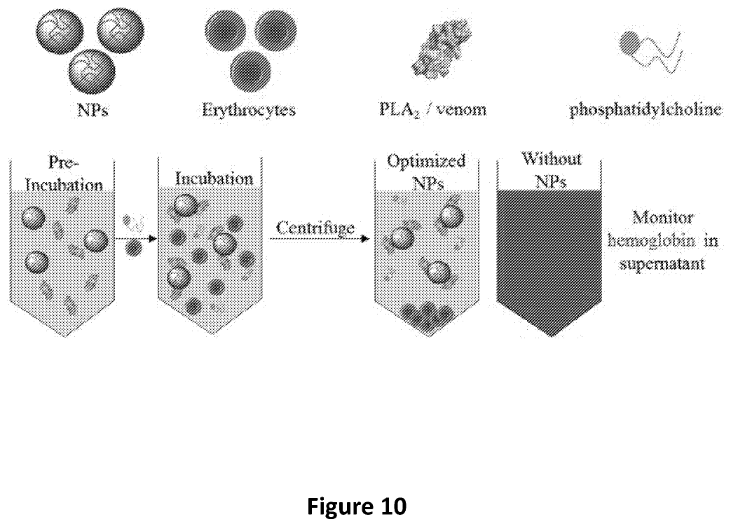

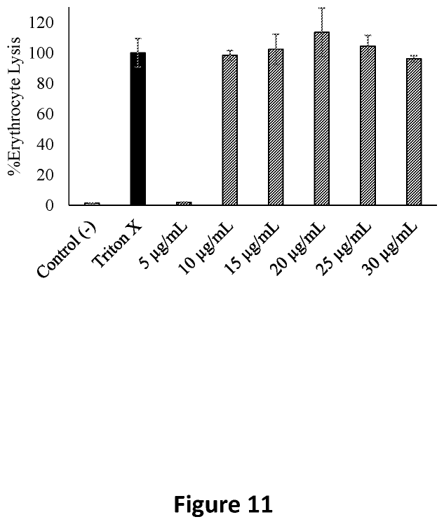

Using NP 1_5 as the lead formulation, a second generation library of NPs were synthesized with systematically varied feed ratios of the four monomers (FIG. 9A, FIG. 9B). The optimization conditions used to evolve the lead formulation from the first generation were made more rigorous by using honey-bee venom instead of snake venom. Honey-bee venom contains two hemolytic principle components: PLA2 and melittin. Melittin is a ˜3 kDa hemolytic pore forming peptide that comprises 50% of the dry mass of honey-bee venom (Zhou J et al., Analytical biochemistry 404.2 (2010): 171-178). The activity of melittin works synergistically with that of PLA2 found in honey-bee venom by releasing phospholipids from lysed cellular membranes allowing for lipid shuttling to occur between melittin and PLA2 (Cajal Y et al., Biochemistry 36.13 (1997): 3882-3893). Thus, monitoring the inhibition of honey-bee venom induced hemolysis through an erythrocyte lysis assay allows for congruent analysis of anti-PLA2 activity and anti-melittin activity. Furthermore, this relatively simplified venom system can be used to test whether or not a single NP composition can inhibit multiple venomous biomacromolecules simultaneously.

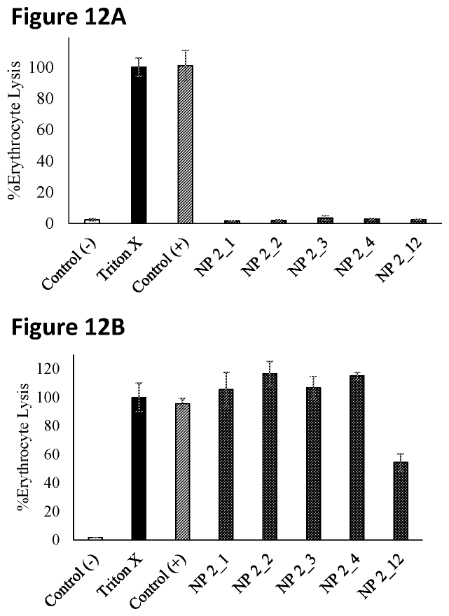

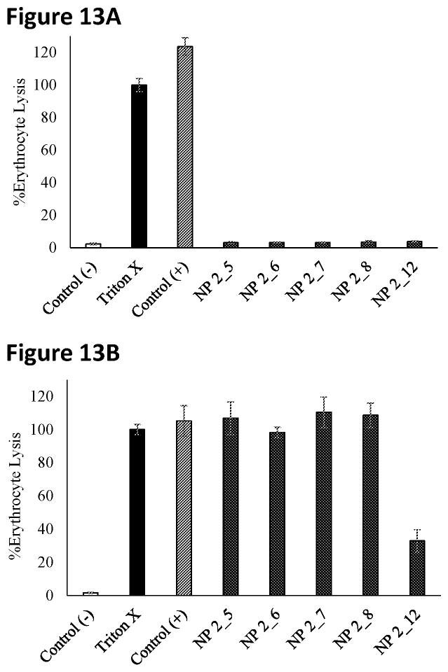

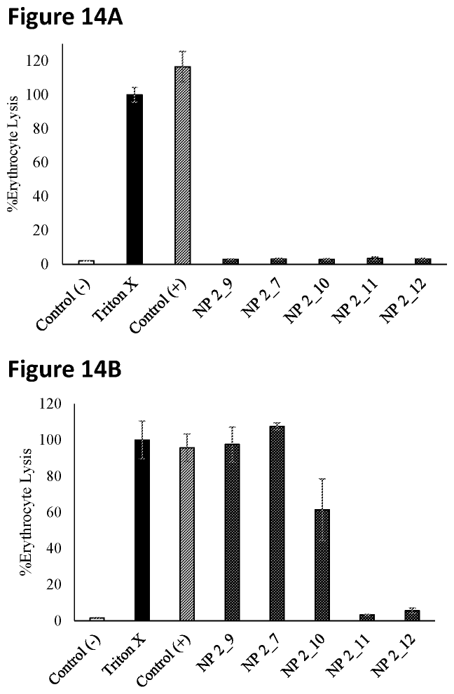

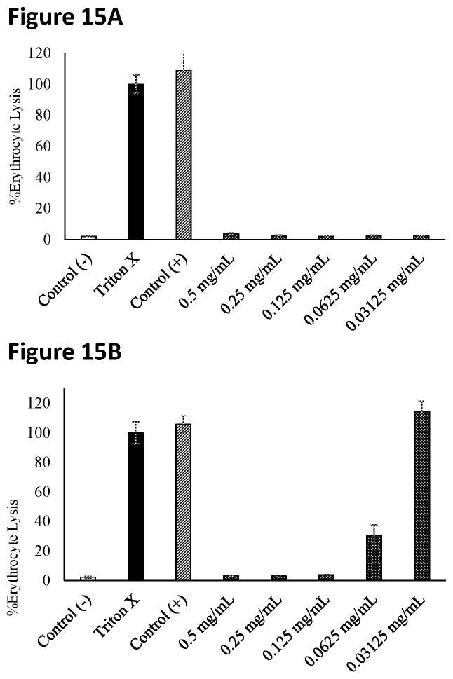

Using a concentration of 10 μg/mL of whole honey-bee venom, the second generation library of NPs were analyzed for their inhibitive properties (FIG. 12A through FIG. 16A). Moreover, the experimental procedure was made more stringent by increasing the final incubation from 15 min to 1 h. Under these conditions, the inhibition of erythrocyte lysis when testing for anti-PLA2 activity by NP 1_5 was overwhelmed. However, it was observed that the amount of cross-linker incorporated into the monomer feed ratio was directly correlated with the inhibition of erythrocyte lysis (FIG. 14B). The NP 2_12, synthesized with the greatest feed ratio of cross-linker (FIG. 16B), was able to inhibit PLA2 induced erythrocyte lysis at concentrations as low as 63 μg/mL (FIG. 16A), and melittin induced erythrocyte lysis at concentrations lower than 0.3 mg/mL (FIG. 16A). While the reasons for this trend in cross-linking are not obvious, it is clear that cross-linking significantly impacts the neutralization of venomous PLA2. More importantly, these results demonstrate that dissimilar biomacromolecules can be neutralized by a single NP formulation, which is a necessary milestone in the pursuit of a broad-spectrum toxin sequestrant.

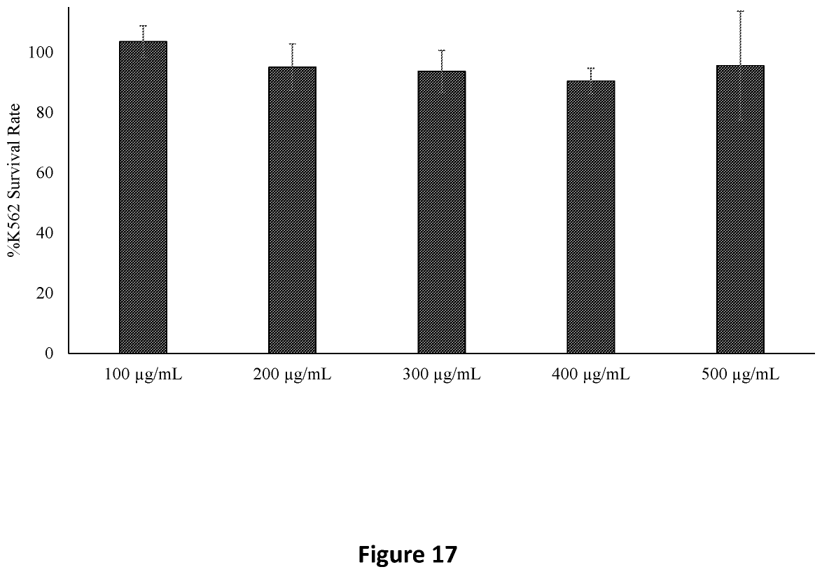

While control experiments were performed on both generations of NPs to ensure that the NPs were not responsible for any observed erythrocyte lysis, the lead NP (NP 2_12) was also subjected to an MTT cell viability test using human immortalized myelogenous leukemia K562 cells. Control experiments were run to establish background absorption at 490 nm in PBS in the presence or absence of NP 2_12 when treated with the MTT dye. However, no cytotoxicity was observed even at NP concentrations as high as 500 μg/mL (FIG. 17).

Following an envenomation, venom proteins are rapidly diluted in the blood stream of the afflicted individual. Thus, a requirement of any antivenom therapeutic is for the therapeutic to have considerable selectivity for the targeted venom proteins over abundant serum proteins.

Exposing NPs to complex biological mixtures results in the rapid adsorption of biomacromolecules onto NPs (Monopoli MP et al., Nature nanotechnology 7.12 (2012): 779-786; Walkey CD et al., Chemical Society Reviews 41.7 (2012): 2780-2799). This rapid adsorption event results in a new and more therapeutically relevant chemical identity termed the protein corona, which can be further divided into two domains: a hard corona and a soft corona (Fleischer CC et al., Accounts of chemical research 47.8 (2014): 2651-2659). The two protein corona domains can be differentiated by the differences in kinetic dissociation rates (koff). The hard corona describes biomacromolecules that slowly dissociate from the NP and the soft corona describes a layer of proteins that rapidly dissociate from the NP or NP⋅hard corona complex. The composition of the hard and soft corona results from the cumulative contribution of a number of physicochemical parameters including size, shape, and the synthetic identity of NP (Lundqvist M et al., Proceedings of the National Academy of Sciences 105.38 (2008): 14265-14270; Nel A E et al., Nature materials 8.7 (2009): 543-557).

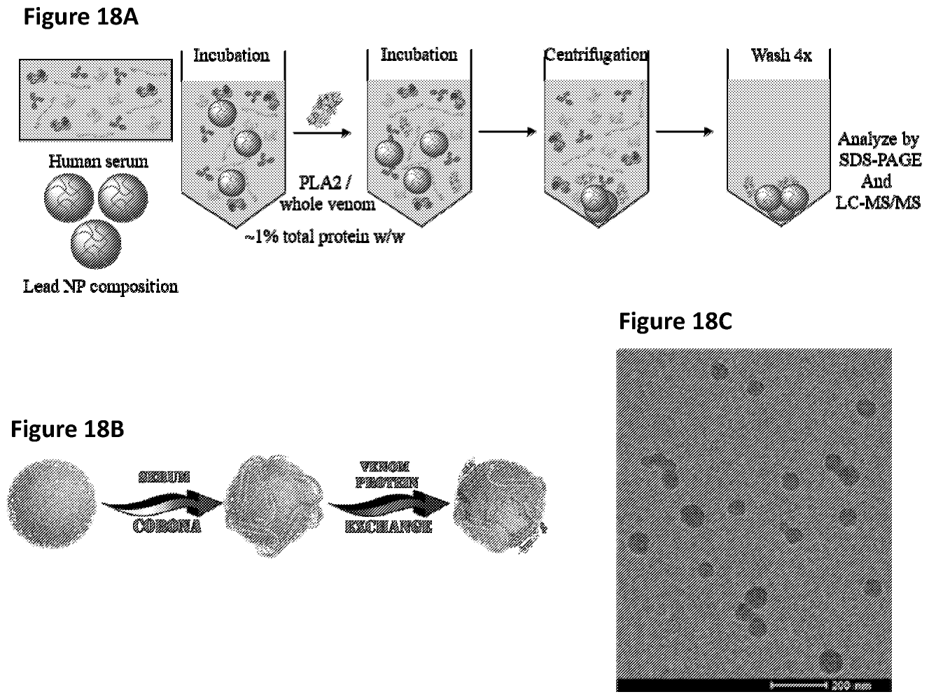

Monitoring the entire protein corona composition has been met with considerable challenges due to the rapidly exchanging soft-corona. However, established methods have been developed for analyzing the composition of the thermodynamic hard-corona via SDS-PAGE and LC-MS/MS (FIG. 18A) (Lundqvist M et al., Proceedings of the National Academy of Sciences 105.38 (2008): 14265-14270). For purposes of venom protein sequestration, it is necessary that venom proteins adsorb within the thermodynamic and slowly-exchanging hard-corona.

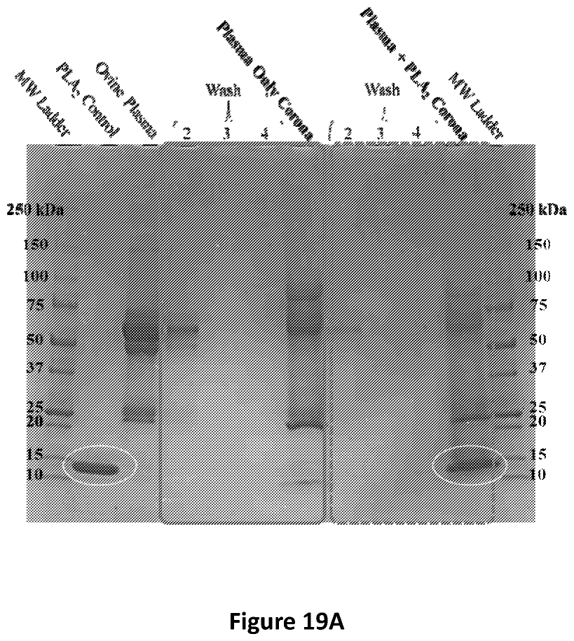

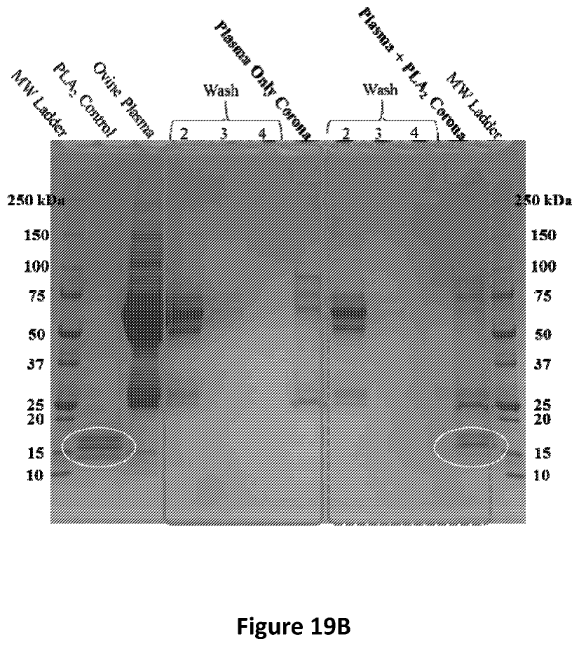

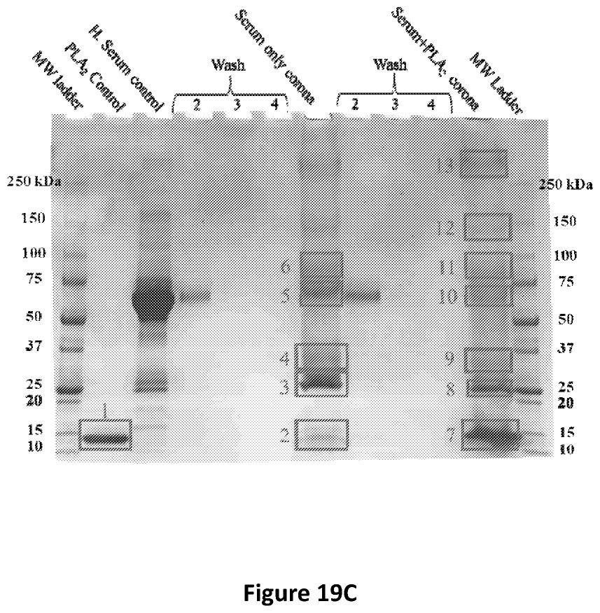

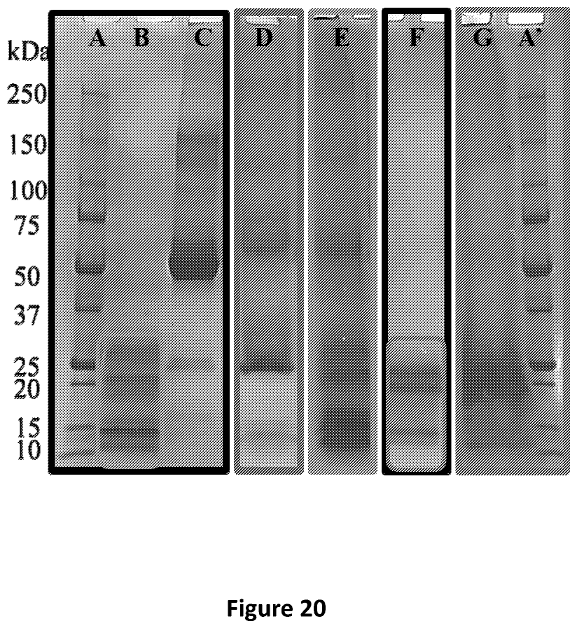

Using concentrated human serum and purified fractions of PLA2 from Naja mossambica venom (˜1% w/w total protein), the hard protein corona of NP 2_12 was analyzed. In the absence of any PLA2, it appears that the majority of the serum proteins have minimal affinity to NP 2_12 (FIG. 19C). However, upon introducing PLA2 after a 5 min pre-incubation in serum, a significant amount of PLA2 appears in the NP protein corona (FIG. 19C). This experiment demonstrates that serum proteins can be displaced by high-affinity venomous PLA2 in the hard corona of the NP, which is a requirement for in vivo venom sequestration/neutralization. This experiment was conducted a second time using bee venom PLA2, which also showed selective association to the NP over ovine plasma proteins (FIG. 19B). These findings demonstrate that a single synthetically optimized NP formulation (NP 2_12) can sequester a variety of PLA2 isoforms and may function as a broad-spectrum venomous-PLA2 sequestrant.

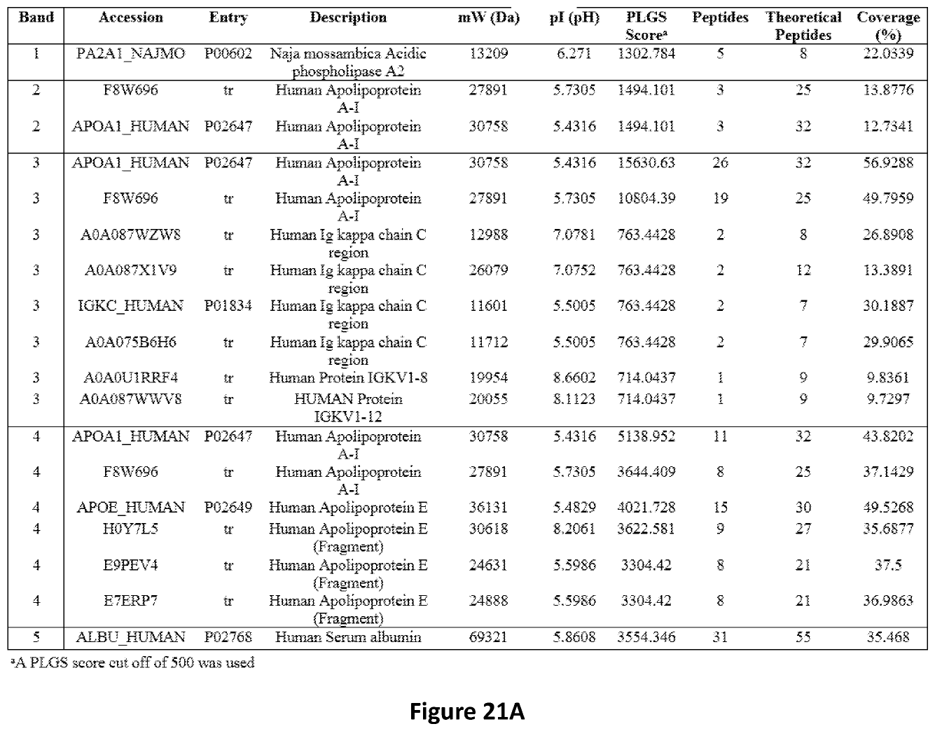

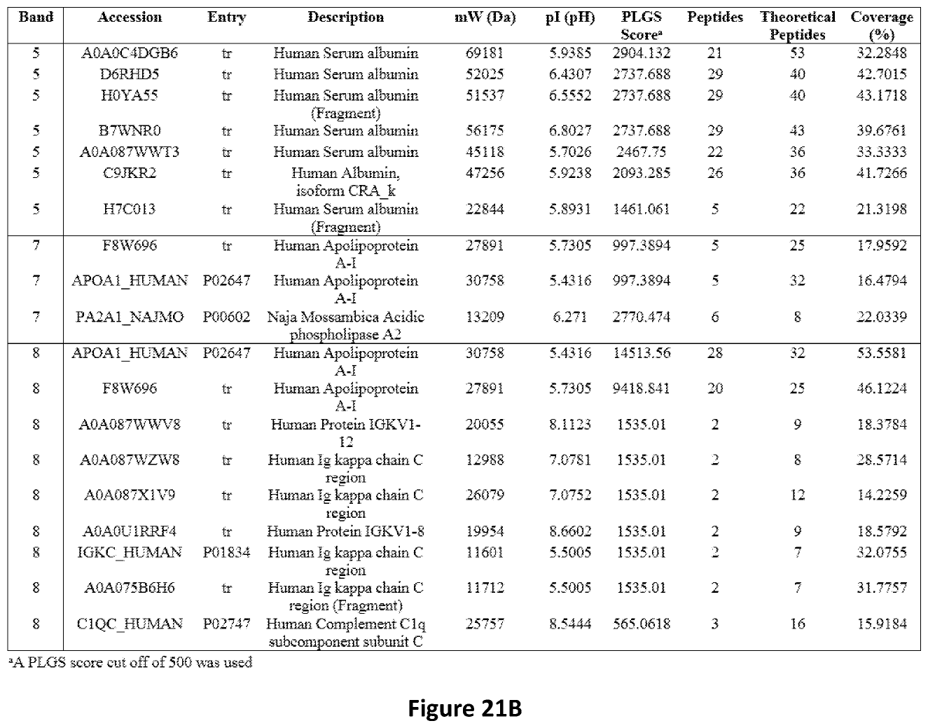

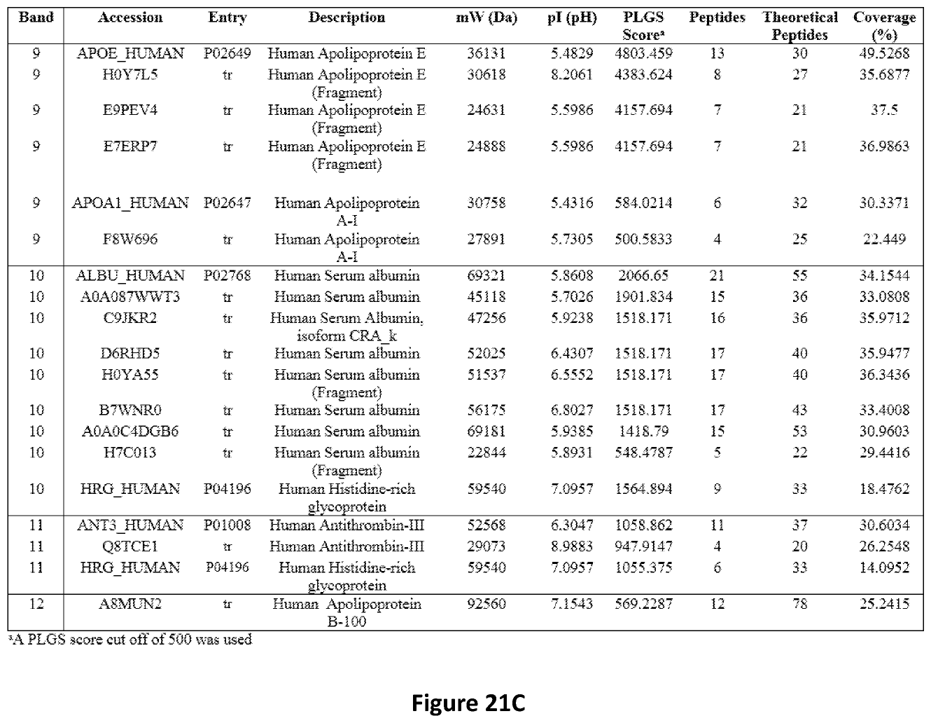

In order to understand the selectivity for PLA2 over other serum proteins, the bands observable by SDS-PAGE were digested and analyzed by mass spectrometry (FIG. 21A through FIG. 21C). Of the nine proteins found in the hard-corona, three were apolipoproteins (Apo-A1, Apo-E, and Apo-B100). In, particular Apo-A1 appears to be the major serum protein bound to NP 2_12. These proteins associate with lipoprotein particles and are important for trafficking lipids throughout the blood-stream (Saito H et al., Progress in lipid research 43.4 (2004): 350-380). This corona profile suggests that the NP is mimicking a natural substrate of PLA2 (lipoprotein particles composed of glycerophospholipids) and explains why this NP composition is capable of sequestering PLA2 with sufficient selectivity over abundant serum proteins. This also explains why NP 2_12 is capable of neutralizing the pore-forming toxin melittin from bee-venom and may act as a broad-spectrum sequestrant for lipid-mediated toxins.

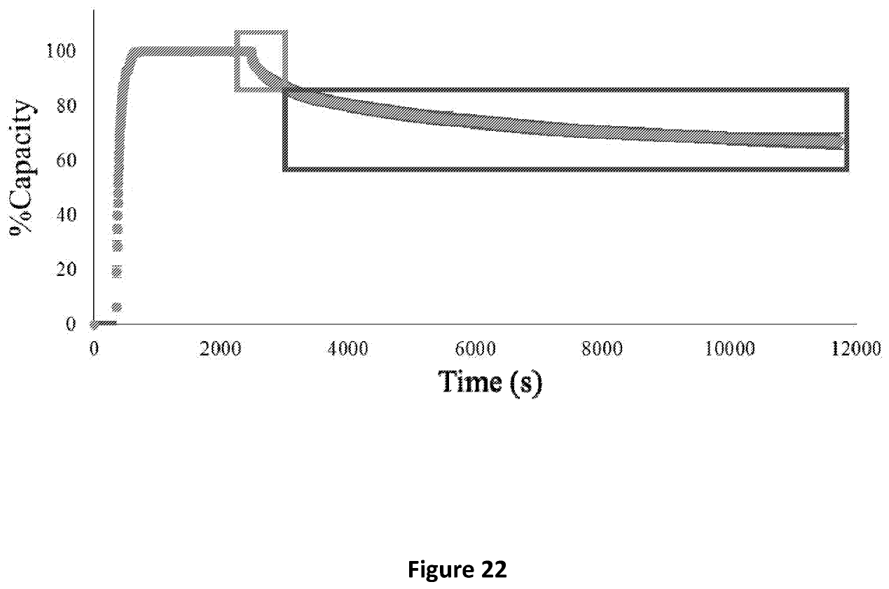

Building on this discovery, the dissociation kinetics were studied for bee venom PLA2⋅NP 2_12. From previous studies aimed at understanding hydrogel-biomacromolecule interactions, it was found that the interaction of NIPAm-based NPs with charged biomacromolecules is dominated by entropically driven processes which can be related to the expulsion of water from the NP hydrogel upon binding (Zeng Z et al., Journal of the American Chemical Society 134.5 (2012): 2681-2690). In the present system, a surface plasmon resonance (SPR) analysis revealed that following a rapid kₒₙ, k_{off} was characterized by rapid desorption of a small (<20%) fraction of absorbed protein (perhaps from the soft corona) followed by a much slower desorption of the remaining (>80%) protein. Under the flow conditions of the SPR experiment, a significant fraction of the bound protein (˜80%, largely PLA2), remains tightly bound. The time needed to release the majority of bound PLA2 from the NP is likely longer than the time needed to clear the PLA2⋅NP 2_12 complex in vivo (FIG. 22). This kinetic data lends credibility to the notion that broad-spectrum sequestration of lipid-mediated toxins may be accomplished via a single optimized NP formulation.

### Example 2: Selectivity Experiments Using Whole Snake Venom Extracts

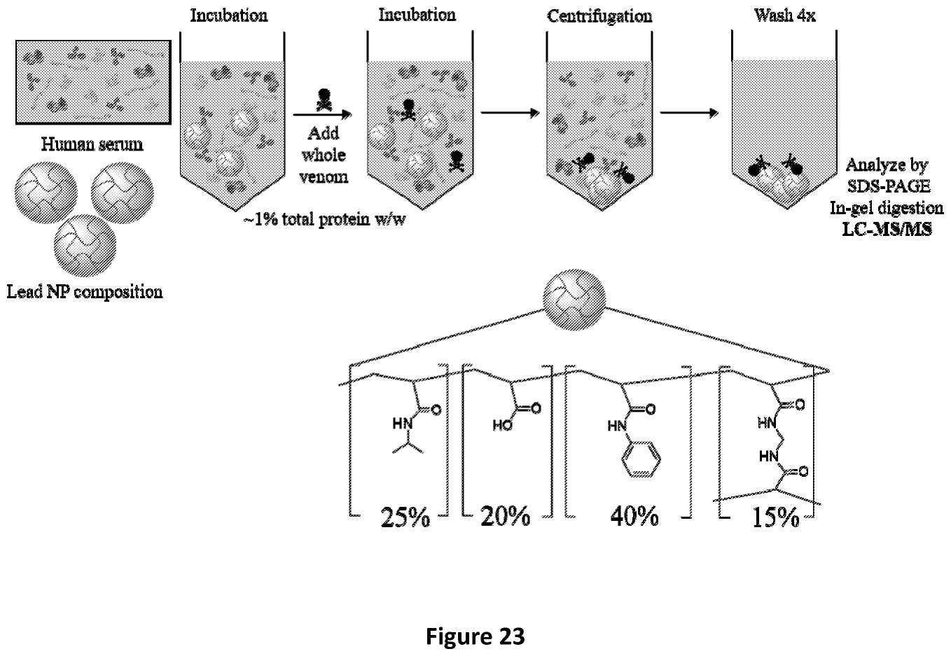

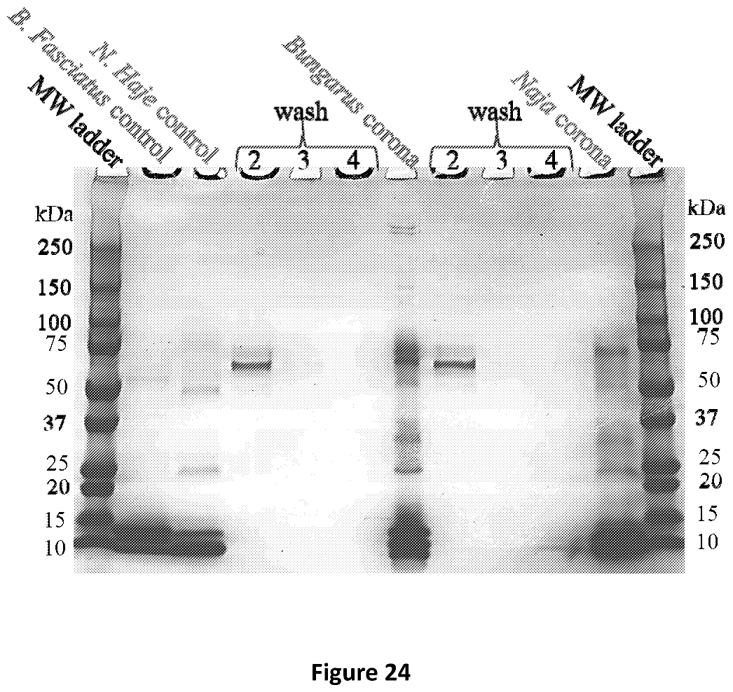

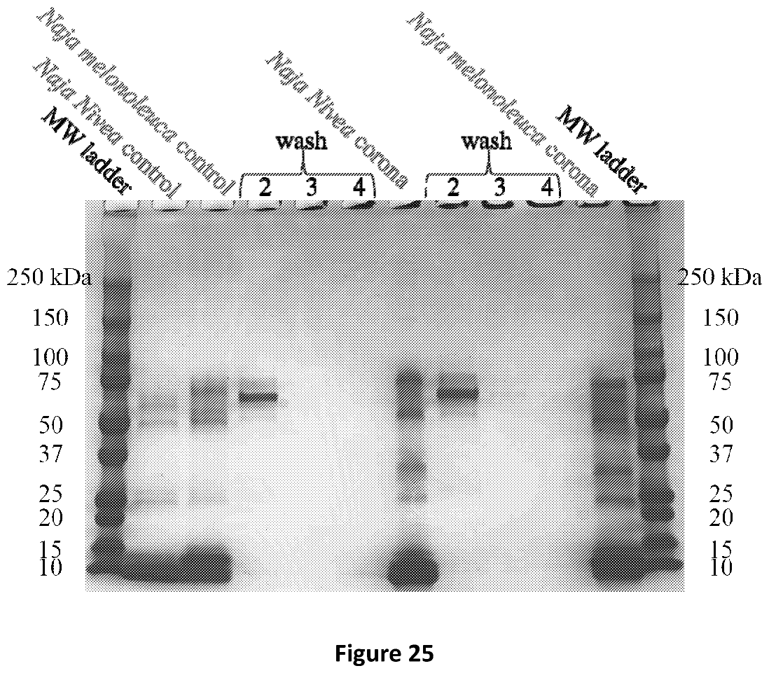

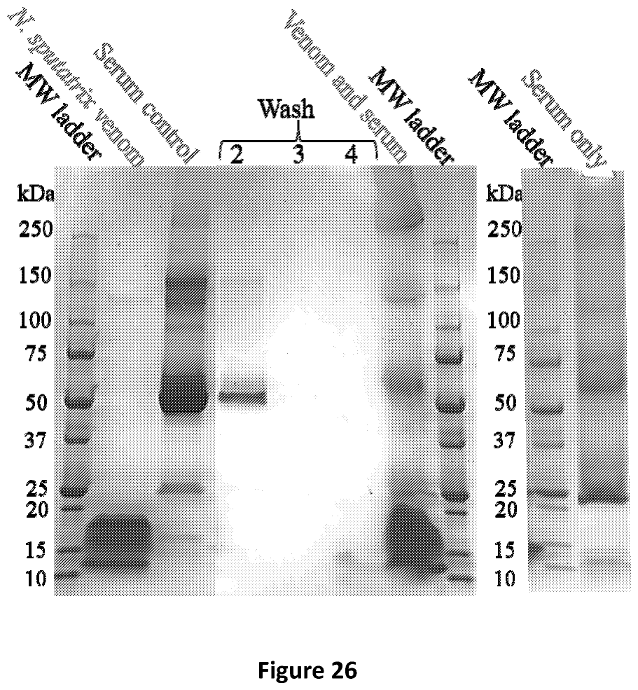

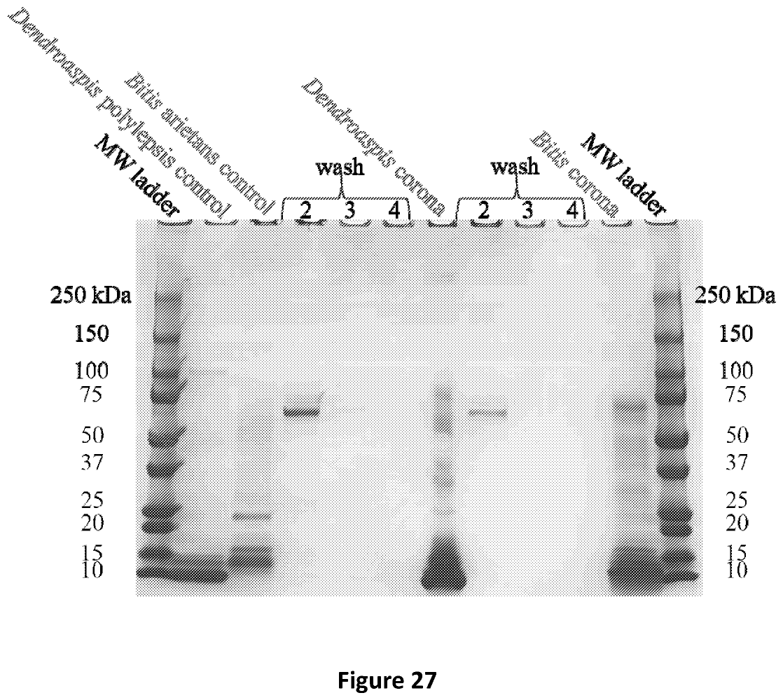

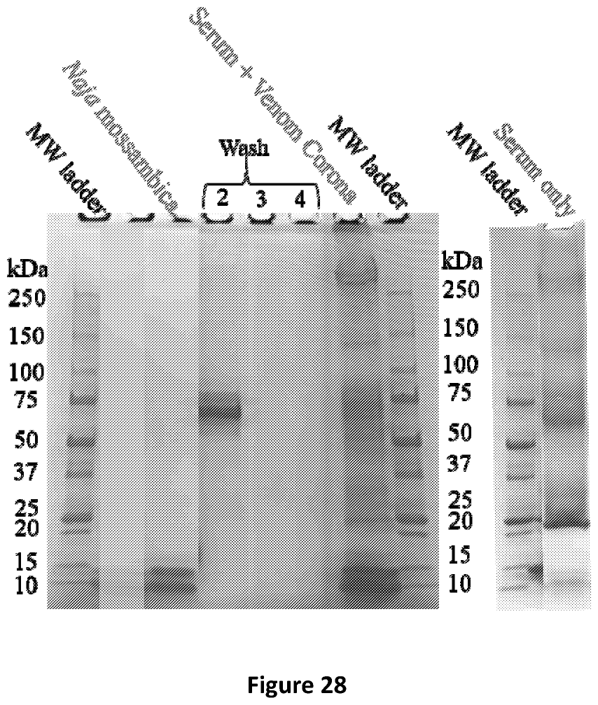

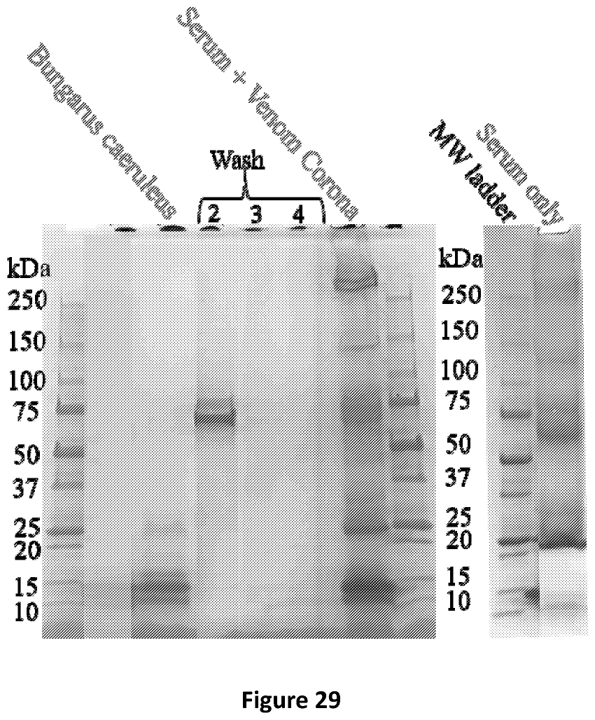

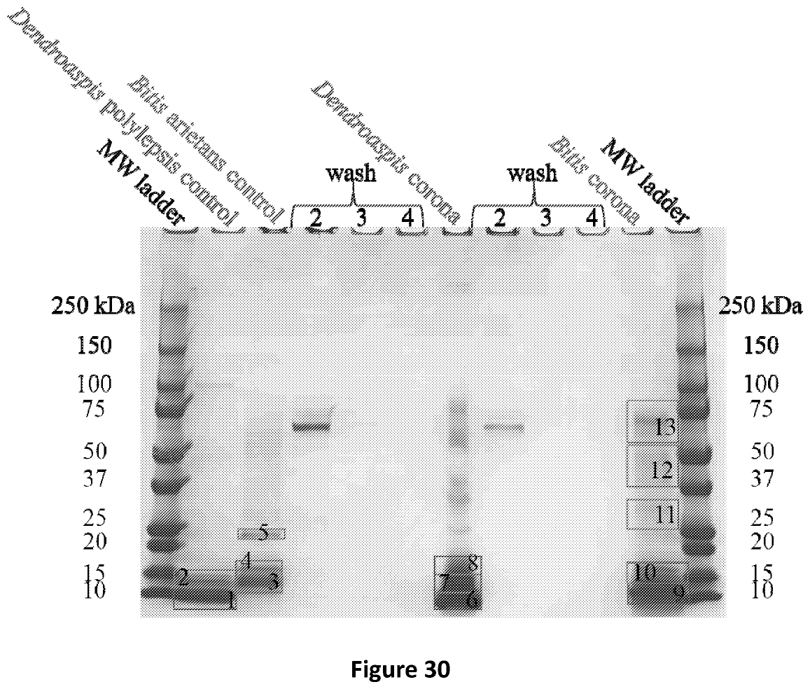

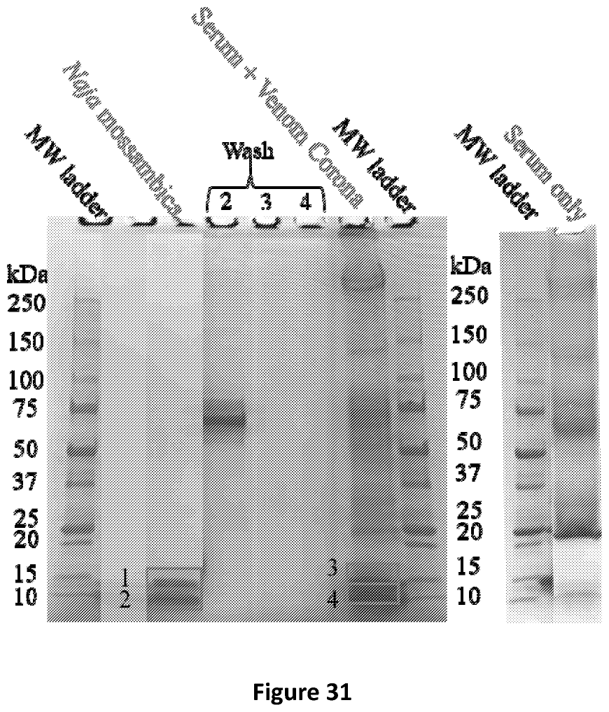

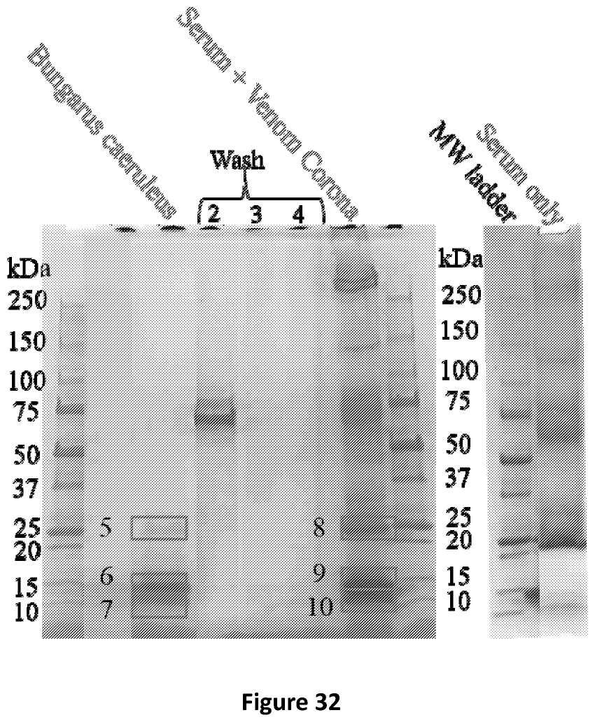

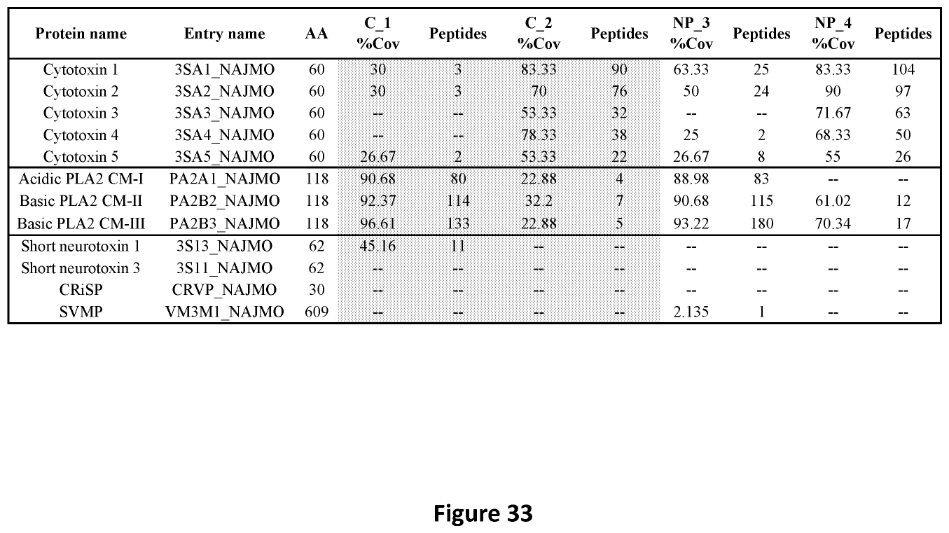

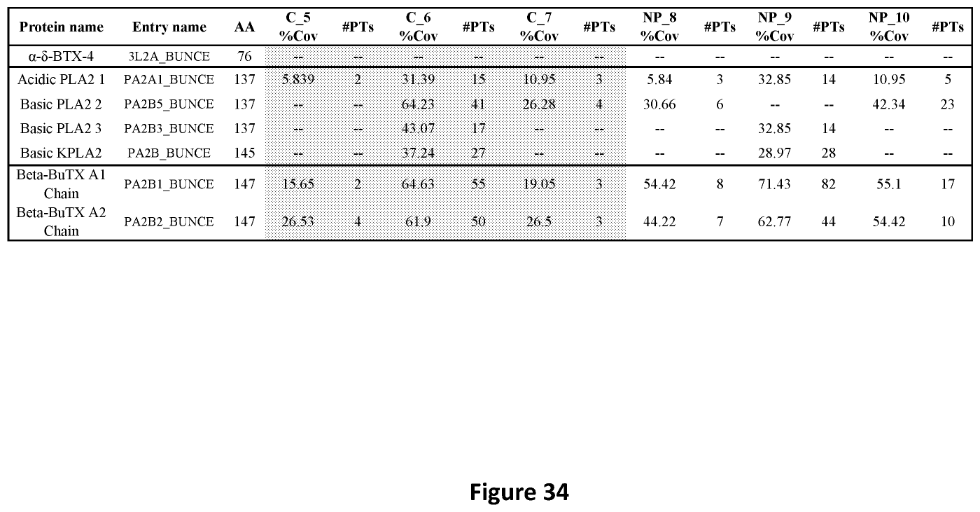

A series of nine different snake venoms were analyzed via the schematic shown in FIG. 23. Four of the experiments were subjected to LC/MS/MS analysis: Naja mossambica, Bungarus caeruleus, Dendroaspis polylepsis, and Bitis arietans (FIG. 30 through FIG. 34).

In all cases, the nanoparticle was able to selectively sequester the toxins over serum proteins, regardless of their protein family. Moreover, the observed results demonstrate that bound serum proteins can exchange with venom toxins, which is necessary for in vivo sequestration and neutralization.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A bandage comprising:
a substrate layer; and
a therapeutic layer comprising a nano-dote composition having at least one component selected from the group consisting of: N-isopropylacrylamide (NIPAm); N-phenylacrylamide (PAA); N-tert-butylacrylamide (TBAm); N,N′-methylenebisacrylamide (Bis); N-acryloyl L-Phenylalinine (APhe); and acrylic acid.

2. The bandage of claim 1, wherein the therapeutic layer comprises a nano-dote composition comprising: N-isopropylacrylamide (NIPAm); N-phenylacrylamide (PAA); N,N′-methylenebisacrylamide (Bis); and acrylic acid.

3. The bandage of claim 2, wherein the therapeutic layer comprises a nano-dote composition comprising between 20% and 30% NIPAm.

4. The bandage of claim 2, wherein the therapeutic layer comprises a nano-dote composition comprising between 30% and 50% PAA.

5. The bandage of claim 2, wherein the therapeutic layer comprises a nano-dote composition comprising between 10% and 20% Bis.

6. The bandage of claim 2, wherein the therapeutic layer comprises a nano-dote composition comprising between 10% and 30% acrylic acid.

7. The bandage of claim 2, wherein the therapeutic layer comprises a nano-dote composition comprising 25% NIPAm, 40% PAA, 15% Bis, and 20% acrylic acid.

8. The bandage of claim 1, wherein the nano-dote composition is a nanoparticle.

9. The bandage of claim 1, wherein the nano-dote composition has affinity to a venomous biomacromolecule.

10. The bandage of claim 5, wherein the biomacromolecule is a three finger toxin (3FTX).

11. The bandage of claim 5, wherein the biomacromolecule is phospholipase A2.

12. The bandage of claim 1, further comprising at least one therapeutic agent.

13. The bandage of claim 12, wherein the at least one therapeutic agent is an enzymatic inhibitor.

14. The bandage of claim 13, wherein the enzymatic inhibitor is a metalloproteinase inhibitor.

15. The bandage of claim 13, wherein the enzymatic inhibitor is a hyaluronidase inhibitor.

16. A method of inhibiting, diminishing, or neutralizing the activity of a venomous biomacromolecule in a subject in need thereof, comprising contacting the subject with the bandage of claim 1.

17. The method of claim 16, wherein the biomacromolecule is a three finger toxin (3FTX).

18. The method of claim 16, wherein the biomacromolecule is phospholipase A2.

19. The method of claim 16, wherein the administration is by topical application.

20. A dispenser comprising:
a body casing;
a reservoir comprising a nano-dote composition having at least one component selected from the group consisting of: N-isopropylacrylamide (NIPAm); N-phenylacrylamide (PAA); N-tert-butylacrylamide (TBAm); N,N′-methylenebisacrylamide (Bis); N-acryloyl L-Phenylalinine (APhe); and acrylic acid;
a plunger; and
a hollow needle fluidly connected to the reservoir.

21. A nano-dote composition comprising at least one component selected from the group consisting of:
N-isopropylacrylamide (NIPAm);
N-phenylacrylamide (PAA);
N-tert-butylacrylamide (TBAm);
N,N′-methylenebisacrylamide (Bis);
N-acryloyl L-Phenylalinine (APhe); and
acrylic acid.

22. The composition of claim 21, comprising:
N-isopropylacrylamide (NIPAm);
N-phenylacrylamide (PAA);
N,N′-methylenebisacrylamide (Bis); and
acrylic acid.

23. The composition of claim 22, comprising between 20% and 30% NIPAm.

24. The composition of claim 22, comprising between 30% and 50% PAA.

25. The composition of claim 22, comprising between 10% and 20% Bis.

26. The composition of claim 22, comprising between 10% and 30% acrylic acid.

27. The composition of claim 22, comprising 25% NIPAm, 40% PAA, 15% Bis, and 20% acrylic acid.

28. The composition of claim 21, wherein the composition is a nanoparticle.

29. The composition of claim 21, wherein the composition has affinity to a venomous biomacromolecule.

30. The composition of claim 25, wherein the biomacromolecule is a three finger toxin (3FTX).

31. The composition of claim 25, wherein the biomacromolecule is phospholipase A2.

32. The composition of claim 21, further comprising at least one therapeutic agent.

33. The composition of claim 32, wherein the at least one therapeutic agent is an enzymatic inhibitor.

34. The composition of claim 33, wherein the enzymatic inhibitor is a metalloproteinase inhibitor.

35. The composition of claim 33, wherein the enzymatic inhibitor is a hyaluronidase inhibitor.

36. A method of inhibiting, diminishing, or neutralizing the activity of a venomous biomacromolecule in a subject in need thereof, comprising administering to a subject a therapeutically effective amount of the nano-dote composition of claim 21.

37. The method of claim 36, wherein the biomacromolecule is a three finger toxin (3FTX).

38. The method of claim 36, wherein the biomacromolecule is phospholipase A2.

39. The method of claim 36, wherein the administration is by topical application.
